(19) United States
(12) Patent Application Publication
(10) Pub. No.: US 2013/0317529 A1
(43) Pub. Date: Nov. 28, 2013

(54) METHODS AND DEVICES FOR METABOLIC SURGERY

(71) Applicants: Steven S. Golden, Menlo Park, CA (US); Pankaj J. Pasricha, Columbia, MD (US); Herber E. Saravia, San Francisco, CA (US)

(72) Inventors: Steven S. Golden, Menlo Park, CA (US); Pankaj J. Pasricha, Columbia, MD (US); Herber E. Saravia, San Francisco, CA (US)

(21) Appl. No.: 13/990,862

(22) PCT Filed: Dec. 2, 2011
(86) PCT No.: PCT/US11/63089

§ 371 Date: Aug. 13, 2013

Related U.S. Application Data
(60) Provisional application No. 61/466,879, filed on Mar. 23, 2011.
(60) Provisional application No. 61/419,665, filed on Dec. 3, 2010.

(51) Int. Cl. A61B 17/22

(52) U.S. Cl. CPC A61B 17/22
USPC 606/159

(57) ABSTRACT

The present disclosure relates to methods and devices for treatment of metabolic and/or gastrointestinal (GI) tract disorders and, more particularly, to surgical methods and devices for the treatment of type 2 diabetes mellitus and Barrett's Esophagus. Even more particularly, the present disclosure pertains to methods and devices for disrupting or removing cells from the GI tract, and methods and devices for harvesting cells from one section of the GI tract and implanting those cells in another section of the GI tract.

### CROSS REFERENCE TO RELATED APPLICATIONS

This application claims the benefit of and priority of U.S. Provisional application Ser. No. 61/419,665, filed Dec. 3, 2010 and U.S. Provisional application Ser. No. 61/466,879, filed Mar. 23, 2011, both of which are hereby incorporated by reference in their entireties.

### FIELD OF THE DISCLOSURE

The present disclosure relates to methods and devices for treatment of metabolic and/or gastrointestinal (GI) tract disorders and, more particularly, to surgical methods and devices for the treatment of type 2 diabetes mellitus and Barrett's Esophagus. Even more particularly, the present disclosure pertains to methods and devices for disrupting or removing cells from the GI tract, and methods and devices for harvesting cells from one section of the GI tract and implanting those cells in another section of the GI tract.

### BACKGROUND

Type 2 diabetes mellitus (T2DM) is a relentless disease affecting over 20 million people in the U.S. alone. The disease stems from the human body's inability to produce insulin or the human body's inability to recognize insulin. Because of the body's inability to produce or recognize insulin, people afflicted with T2DM do not properly utilize glucose for energy.

Traditionally, T2DM has been treated through diet, exercise and/or medication. Recently, some medical studies have indicated that this disease may be treated with bariatric surgeries, such as those that have been commonly used to reduce the size of the stomach in connection with the treatment of obesity. In light of these medical studies, metabolic surgical treatments for non-obese patients have been developed. Such surgical treatments typically entail a rearrangement of whole sections of the GI tract, for example, the transposition of one section of small bowel (e.g. ileum) to a more proximal section of the small bowel (e.g. jejunum). The objective of such transposition surgeries is to modify the hormones which are produced in the proximal section of the GI tract with the intent of improving glucose homeostasis. It has been shown that such transposition surgeries can result in greater production of hormones in the proximal section of the GI tract that are associated with mediating diabetes. Such hormones may include Glucagon-like peptide-1 (GLP-1) and peptide-YY. Though these bariatric surgical techniques are sometimes referred to as “minimally invasive”, they are still very traumatic and invasive surgeries involving anatomical reconstruction, oftentimes requiring incisions through multiple layers of tissue, general anesthesia and hours to perform. In addition, these types of surgeries are not easily reversed since it involves significantly modifying anatomical structures from the natural state of the body.

Barrett's Esophagus is a precancerous condition of the lower esophagus that affects more than 3 million people over the age of 50. It is characterized by the presence of specialized intestinal metaplasia which is an abnormal cell formation on the mucosal layer of the esophagus. This has been found to be a precancerous condition. Patients with Barrett's Esophagus are 30-125 times more likely to develop adenocarcinoma (esophageal cancer). The cause of Barrett's esophagus is gastric reflux, particularly silent reflux which has been left untreated over a long period of time. Continual exposure of the esophageal lining to the stomach acids causes damage and eventually genetic changes to these cells, leading to the formation of an abnormal esophageal lining.

Currently, Barrett's is treated by removing or destroying the affected mucosal tissue. In one common treatment RF energy is used to ablate the mucosal lining of the esophagus. Such ablation methods, however, have some drawbacks in that they are expensive, time consuming, and destroys the tissue, making it impossible to retrieve and analyze in the lab. Other treatments include mechanical excision of the afflicted tissue. Such procedures may include Endoscopic Mucosal Resection (based on methodology for removing colon polyps), which is a time consuming method of removing small isolated spots of tissue and does not lend itself to quickly and efficiently excising larger areas.

### SUMMARY

In one aspect of the present disclosure, an apparatus for removing cells from an inner wall of a gastrointestinal tract, includes a cell manipulator defining an inner chamber and having at least one opening in communication with the inner chamber. The apparatus also includes at least one tissue disruption surface positioned adjacent to the opening. The tissue disruption surface is configured to separate/remove, for example cut or scrape, the cells of the inner wall of the gastrointestinal tract.

In another aspect of the present disclosure, a method of removing cells from an inner wall of a gastrointestinal tract including the step of contacting target tissue with a cell manipulator and applying suction to the target tissue. The method also includes the steps of disrupting cells from the inner wall of the gastrointestinal tract with the cell manipulator and evacuating said cells from the gastrointestinal tract with suction.

In a further aspect of the present disclosure, a method for treating metabolic conditions includes harvesting cells from a first section of a gastrointestinal tract and implanting at least some of the cells in a second section of the gastrointestinal tract.

In another aspect, a method for treating metabolic conditions that includes accessing a first section of the gastrointestinal tract (for example, the small intestine) and harvesting cells from a mucosal or submucosal of first section of the gastrointestinal tract. The method further includes accessing a second portion of the gastrointestinal tract (for example the small intestine) and implanting the at least some of the cells in the second portion of thegastrointestinal tract.

In yet another aspect, an apparatus for separating cells from the mucosa or submucosa of the small intestine includes an outer member including a proximal end portion and a closed distal end portion. The closed distal end portion defines an inner cavity and has an opening in communication with the inner cavity. The apparatus also includes an inner member located within the cavity of the distal end portion of the outer member. The inner member includes a cell disruption element that is adapted to access cells through the opening of the closed distal end of the outer member. The cell disruption element moves relative to the opening to disrupt the cells.

In a further aspect, an apparatus for removing cells from the gastrointestinal tract includes an access device that has a proximal end portion, a distal end portion and a passageway therethrough. The apparatus also includes a cell manipulator received within the passageway of the access device and that is distally advanceable so that a portion of the cell manipulator is advanced beyond the distal end portion of the access device. The cell manipulator includes a cell disruption element for disrupting cells of the gastrointestinal tract.

In another aspect, an apparatus for removing cells from the inner wall of the gastrointestinal tract includes a cell manipulator having a closed distal end portion and a cavity defined by the closed distal end portion. The cell manipulator includes a side opening in the closed distal end portion wherein the side opening is in communication with the cavity. The apparatus also includes a cell disruption surface adjacent the side opening The cell disruption surface is adapted to access at least a portion of the inner wall of the gastrointestinal tract through the side opening.

In yet a further aspect, a method of treating the esophagus includes accessing a selected portion of the esophagus and placing a cell manipulator at the selected portion of the esophagus. The cell manipulator is employed to disrupt selected cells to remove the selected cells from the esophagus.

In yet another aspect, a method of treating a lumen of a human body includes harvesting selected cells from a first section of a lumen of the human body and implanting at least some of the selected cells in a second section of the lumen.

In other aspects methods and apparatus are provided for removing tissue/cells from the inner wall of the gastrointestinal tract using scope-mounted cell manipulators combined with suction and collection of the tissue/cells for pathologic analysis.

### BRIEF DESCRIPTION OF THE FIGURES

In the course of this description, reference will be made to the accompanying drawings, wherein:

FIG. 1 is a schematic illustration of a GI tract;

FIG. 2 is a flowchart of one embodiment of a method of treating a metabolic condition in accordance with the present disclosure;

FIG. 3 is a flowchart of another embodiment of a method of treating a metabolic condition in accordance with the present disclosure;

FIG. 4 is a side view of one embodiment of an access device;

FIG. 5 is an illustration of a GI tract having the access device of FIG. 4 inserted therein;

FIG. 6 is another illustration of a GI tract having the access device of FIG. 4 inserted therein;

FIG. 7 is a side view of one embodiment a system that may be used to access a section of the GI tract and disrupt cells of a GI tract;

FIG. 8 is an illustration of a section of a GI tract having one embodiment of a cell manipulator located therein;

FIG. 9 is a perspective view of one embodiment of a cell manipulator;

FIG. 10 is an elevation view of the cell manipulator of FIG. 9 shown within a section of a GI tract;

FIG. 11 is a perspective view of another embodiment of cell manipulator;

FIG. 12 is a perspective view of one embodiment of the inner member of the cell manipulator of FIG. 11;

FIG. 13 is a perspective view of another embodiment of a cell manipulator;

FIG. 14 is a perspective view of one embodiment of the inner member of the cell manipulator of FIG. 13;

FIG. 15 is a perspective view of another embodiment of a cell manipulator;

FIG. 16 is a perspective view of another embodiment of a cell manipulator;

FIG. 17 is a perspective view of the cell manipulator of FIG. 16 shown within a section of a GI tract;

FIG. 18 is a perspective view of another embodiment of a cell manipulator shown within a GI tract;

FIG. 19 is a perspective view of another embodiment of a cell manipulator shown within a GI tract;

FIG. 20 is a perspective view of another embodiment of a cell manipulator shown within a GI tract;

FIG. 21 is a perspective view of one embodiment of a tissue guard shown within a GI tract;

FIG. 22 is a cross-sectional view of the tissue guard of FIG. 21 shown within a GI tract;

FIG. 23 is a perspective view of one embodiment of a cell applicator shown within a GI tract;

FIG. 24 is a perspective view of one embodiment of a system that may be used to apply cells to a GI tract;

FIG. 25 is a perspective view the cell manipulator of FIG. 18 shown within an esophagus;

FIG. 26 is a perspective view of another embodiment of a cell manipulator shown within an esophagus;

FIG. 27 is a perspective view the cell manipulator of FIG. 20 shown within an esophagus;

FIG. 28 is a perspective view of another embodiment of a cell manipulator shown within an esophagus;

FIG. 29 is a perspective view the cell manipulator of FIG. 19 shown within an esophagus;

FIG. 30 is a perspective view of the tissue guard of FIG. 21 shown within an esophagus;

FIG. 31 is a cross-sectional view of the tissue guard of FIG. 21 shown within the esophagus;

FIG. 32 is a perspective view of another embodiment of a tissue guard shown within a GI tract;

FIG. 33 is a cross-sectional view of another embodiment of a tissue guard shown within a GI tract;

FIG. 34 is a perspective view of another embodiment of a cell manipulator;

FIG. 35 is perspective view of the reverse side of the cell manipulator of FIG. 34;

FIG. 36 is an exploded view of the cell manipulator of FIG. 34;

FIG. 37 is a cross-sectional view of the cell manipulator of FIG. 34;

FIG. 38 is a cross-sectional view of the cell manipulator of FIG. 34 shown inside a GI tract and acting upon tissue/cells;

FIG. 39 is a perspective view of an alternate embodiment of a cell manipulator;

FIG. 40 is a cross-section view of another embodiment of a cell manipulator;

FIG. 41 is a perspective view of the cell manipulator shown in FIG. 40;

FIG. 42 is a perspective view of an alternate embodiment of a tissue disruption element;

FIG. 43 is a perspective view of another embodiment of a tissue disruption element;

FIG. 44 shows a perspective view of another embodiment of a cell manipulator in a retracted state;

FIG. 45 is a perspective view of the cell manipulator of FIG. 44 shown in an extended state;

FIG. 46 is a perspective view of another embodiment of a cell manipulator;

FIG. 47 is an exploded view of the cell manipulator shown in FIG. 46;

FIG. 48 is a cross-section view of the distal portion of the cell manipulator of FIG. 46;

FIG. 49 is a cross-section view of the proximal portion of the cell manipulator of FIG. 46;

FIG. 50 is a cross-section view of the cell manipulator of FIG. 46 inserted inside a GI tract;

FIG. 51 is an axial cross-section view of the proximal end of cell manipulator of FIG. 46;

FIG. 52 is a schematic of a tissue/cell manipulator system including the suction and tissue collection aspects;

FIG. 53 is a cross-section view of an embodiment of the collection device shown in FIG. 52;

FIG. 54 is a perspective view of another embodiment of a cell applicator shown within the GI tract;

FIG. 55 is a perspective view of a cap-and-needle embodiment of a cell applicator; and

FIG. 56 is a cross section view of the cap-and-needle embodiment of a cell applicator of FIG. 55 shown inside a GI tract.

### DETAILED DESCRIPTION

Although, detailed embodiments of the present invention are disclosed herein, it will be understood that the disclosed embodiments are merely exemplary of the invention and various aspects thereof, which may be embodied in various forms. Therefore, specific details disclosed herein are not to be interpreted as limiting, but as illustrative and informative for a person skilled in the subject matter.

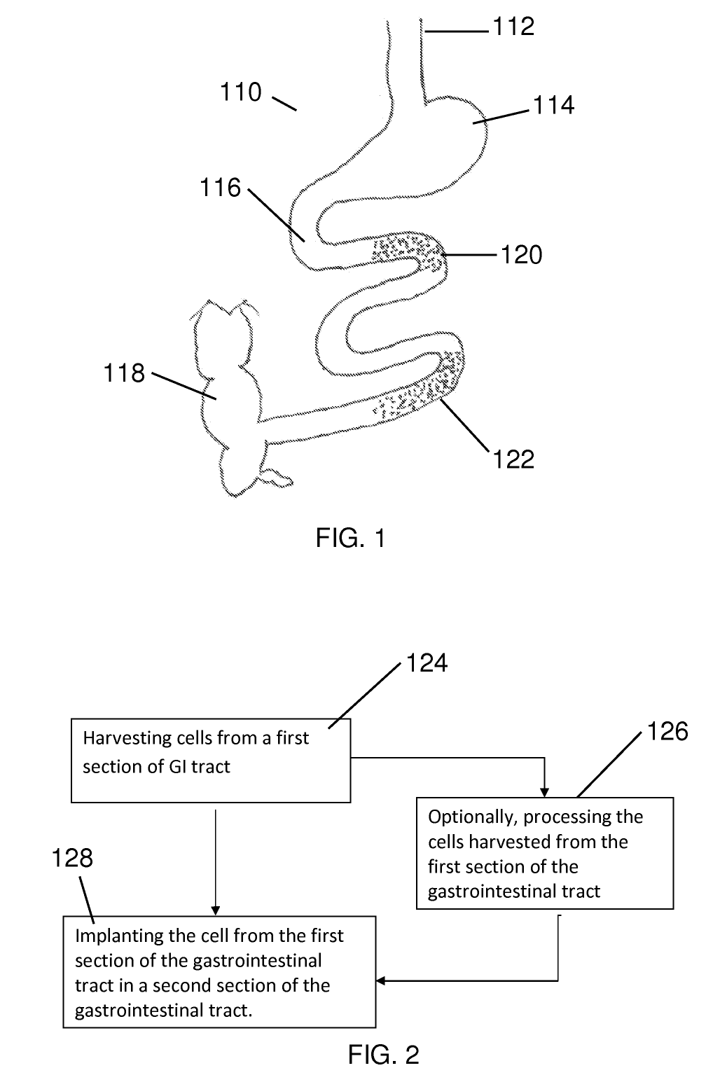

FIG. 1 generally shows a gastrointestinal (GI) tract 110 of a human body. GI tract 110 includes a number of interconnected lumens or portions. Briefly, the GI tract includes the esophagus 112, the stomach 114, the small intestine 116 and the large intestine 118. The small intestine 116 includes the jejunum 120 and ileum 122.

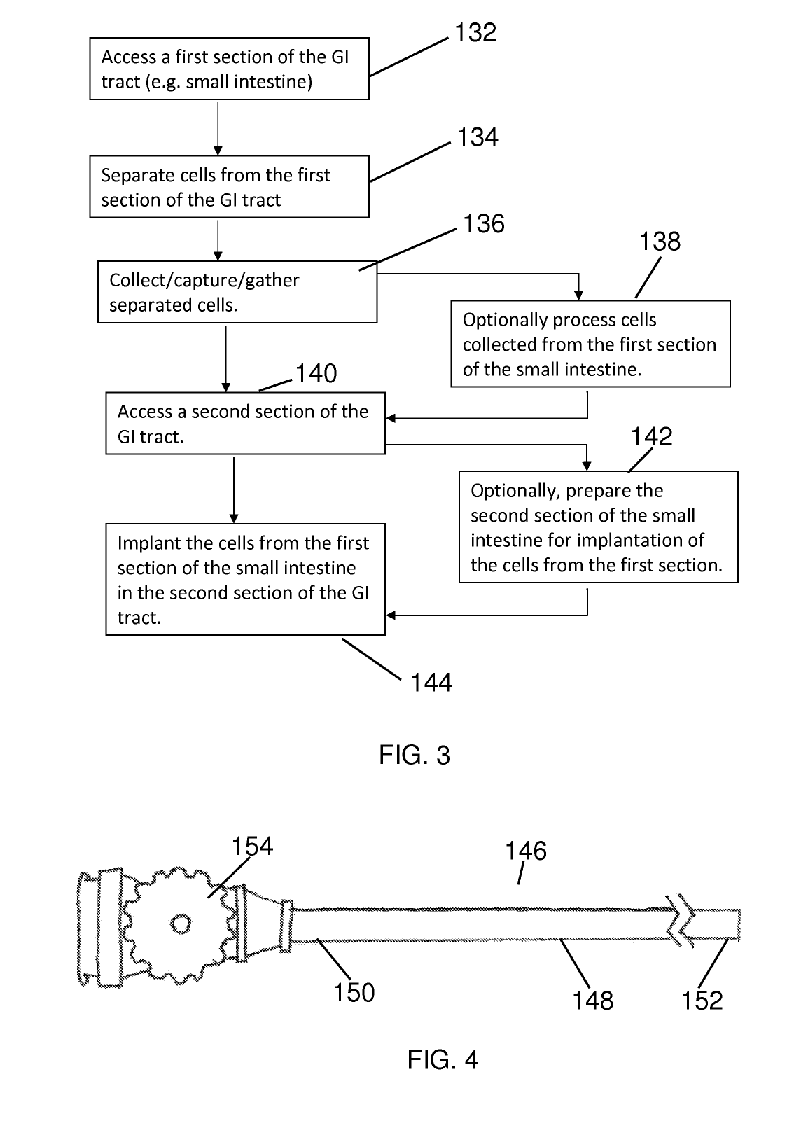

FIGS. 2 and 3 are flow diagrams illustrating examples of surgical methods according to the present disclosure that may be used to treat metabolic disorders, such as diabetes, and more specifically T2DM. Referring to FIG. 2, at 124, the surgical method includes harvesting or otherwise obtaining tissue cells from a first section of the GI tract, such as but not limited to the ileum. In one embodiment, harvesting tissue cells includes separating, resecting or otherwise removing cells from the first section of the GI tract and collecting or otherwise capturing the separated cells. The cells may be separated from the first section of the GI tract by cutting, scraping or otherwise disrupting the cells, or by any other suitable methods and techniques that separate cells from the first section of the GI tract. The cells may be separated from the first section of the GI tract as individual cells, small groups of cells and/or large groups of cells or tissue layers.

The separated tissue cells may be collected, captured or gathered by any suitable cell collection method and devices. For example, the separated cells may be collected under a suction force wherein the separated cells are suctioned into a collection device or chamber. In another embodiment, the separated cells may be collected by a device in which the cells adhere to or become entangled with the device. For instance, the collection device may include a plurality of bristles wherein the separated cells adhere or become entangle in the bristles. In still other embodiments, the separated cells may be collected by a device that includes a grasping member, such as forceps.

The cells harvested from the first section of the GI tract maybe immediately moved to another or second section of the GI tract for implantation or may be completely removed or retrieved from the GI tract prior to implantation into the second section of the GI tract. Further, if desired, the cells harvested from the first section of the GI tract may be subjected to any variety of cell processing procedures, as shown at 126, prior to implantation into the second section of the GI tract. For example, the cells may be subjected to separation processes to separate selected cells from other tissue or cells, washing procedures to remove blood, fat or other cells, concentrating procedures and/or other cell processing procedures. The cells harvested from the first section of the GI tract may, as desired, be suspended in a preservative or other solution for processing storage and/or subsequent implanting. The cells harvested from the first section of the GI tract also may be attached to a support matrix or support surface for later implantation. Furthermore, the cells harvested from the first section of the GI tract also may be subjected to culturing for increasing cell population, enhancing the viability or promoting cell implantation.

At 128, the cells harvested from the first section of the GI tract, and optionally processed, are then implanted in a second section of the GI tract, such as but not limited to the jejunum. The cells may be implanted in the GI tract by any suitable method known in the art. For example, the cells may be grafted, sutured, or glued. Alternatively, the cells may be attached to a support matrix, which is implanted in the second section of the GI tract. Further, the cells and/or the second section of the GI tract may be treated with therapeutic agents and/or other agents that assist or enhance the implantation of the cells in the second section of the GI tract.

In one embodiment of a surgical method for treating a metabolic disorder, cells are harvested from the ileum of the small intestine and implanted in the jejunum of the small intestine. This method may be particularly useful for, but not necessarily limited to, treatment of T2DM. Without being limited to any specific scientific theory, it is believed that harvesting mucosa or submucosa cells and, more particularly L-cells located in the mucosa or submucosa of the ileum of the small intestine, and implanting such cells in the jejunum of the small intestine will result in modification of the hormones produced within the jejunum. In particular, it is believed that implanting such cells in the jejunum will provide a greater production of diabetes mediating hormones, such as GLP-1 and peptide YY, in the jejunum, and will result in improved glucose homeostasis.

Referring now to the method illustrated in FIG. 3, at 132 of the flow diagram, a first section of the GI tract such as the small intestine is accessed. The first section of the small intestine may be, for example, the ileum or the jejunum. In one embodiment, the first section of the small intestine is accessed using common endoscopic surgical techniques. The endoscopic surgical techniques may include accessing the first section of the small intestine through natural orifices, such as the anus or the mouth. In alternative embodiments, the endoscopic surgical techniques may include accessing the first section of the small intestine through incisions, preferably for thoracoscopic-type access, in the abdomen.

Once access to the small intestine has been obtained, referring now to 134 of the flow diagram, the cells from a selected portion of the first section of the GI tract are separated or otherwise resected from the selected portion of the first section of the GI tract. The cells may be separated/removed from the selected portion by any suitable method or technique, including but not limited to, scraping, cutting, shaving or otherwise disrupting the cells in order to separate or extricate the cells from the selected portion of the first section. At 136, the separated cells are then collected or captured for transplant or removal from the GI tract at some desired time. In one embodiment the cells are separated from the GI tract and collected/captured at the same time or during the same procedure. Optionally, as shown at 138, the separated and collected cells may be subjected to cells processing procedures, such as any of the cell processing procedures disclosed herein or any other suitable cell processing procedures.

At 140, a second section or location of the GI tract is accessed. For example a second section of the small intestine is accessed. The second section of the small intestine may be in relatively close proximity to or spaced apart from the first section. For example, the first and second section both may be located in the jejunum or both may be located in the ileum. In another example, the first and second section may be located in any section of the GI tract,where the first and second section are spaced some distance from one another in the GI tract. In alternative embodiments, the first and second section of the small intestine may be spaced some distance from one another. For example, one of the first or the second sections may be located in the jejunum and the other of the first and second sections may be located in the ileum. Preferably, the section from where the cells are taken is the ileum and the section into where they implanted is the jejunum. Similar to accessing the first section, the second section of the small intestine may be accessed using endoscopic surgical techniques. The endoscopic surgical techniques may include accessing the second section of the small intestine through natural orifices, such as the anus or the mouth. In alternative embodiments, the endoscopic surgical techniques may include accessing the second section of the small intestine through incisions in the abdomen, such as relatively small thoracoscopic incisions.

After the second section has been accessed, optionally, the second section may be prepared to accept implantation of the collected cells, as shown at 142.

Preferably, the second section is prepared in such a manner that promotes attachment of the cells collected from the first section and/or promotes growth and proliferation of such cells. In one embodiment, the second section of the small intestine is prepared by cutting, scraping or otherwise disrupting tissue of the second section. For example, preparing the second section may entail cutting or scraping (physically or chemically) the inner wall of the second section of the small intestine. In one embodiment, preparing the second section includes removing the mucosal layer from the second section. In alterative embodiments, the second section may be prepared by applying a therapeutic agent, graft-promoting agent, and/or other bioactive agents to the second section. In further embodiments, the second section may be prepared by applying tissue adhesives to the second section. In still other embodiments, preparing the second section may include any combination of the above mentioned preparation techniques, such as cutting or scrapping and application of an adhesive and/or therapeutic agent.

Referring now to 144 of the flow diagram of FIG. 3, the collected cells are then implanted in the second section of the small intestine. The collected cells may be implanted or grafted by any suitable cell implantation technique known in the art. For example, implantation of the collected cells may include the use of tissue or cell adhesives, therapeutic agents or sutures.

In a further embodiment and optionally, when the second section of the inner wall is prepared by implantation by disrupting cells from the inner wall of the second section, such disrupted cells may be collected, optionally processed and implanted in another part or location of the GI tract. For example, disrupted cells collected from the second section of the small intestine may be implanted in the first section of the small intestine.

Accessing the GI tract

In the methods disclosed herein, any suitable technique known in the art may be used to access the first and second sections of the GI tract. For example, the accessing techniques may include, but are not limited to, access approaches through a natural body orifice such as the anus, mouth or nose. Such approaches include transoral and transanal approaches. Access approaches through natural body orifices typically do not require skin incisions or general anesthesia. Furthermore, these approaches typically require less time to perform and can reduce risk, trauma and discomfort to the patient, and may reduce recovery times. The access approaches, however, are not limited to those through a natural body orifice and other more invasive approaches, such as endoscopic approaches through incisions in the abdomen and open abdominal surgeries, may be employed.

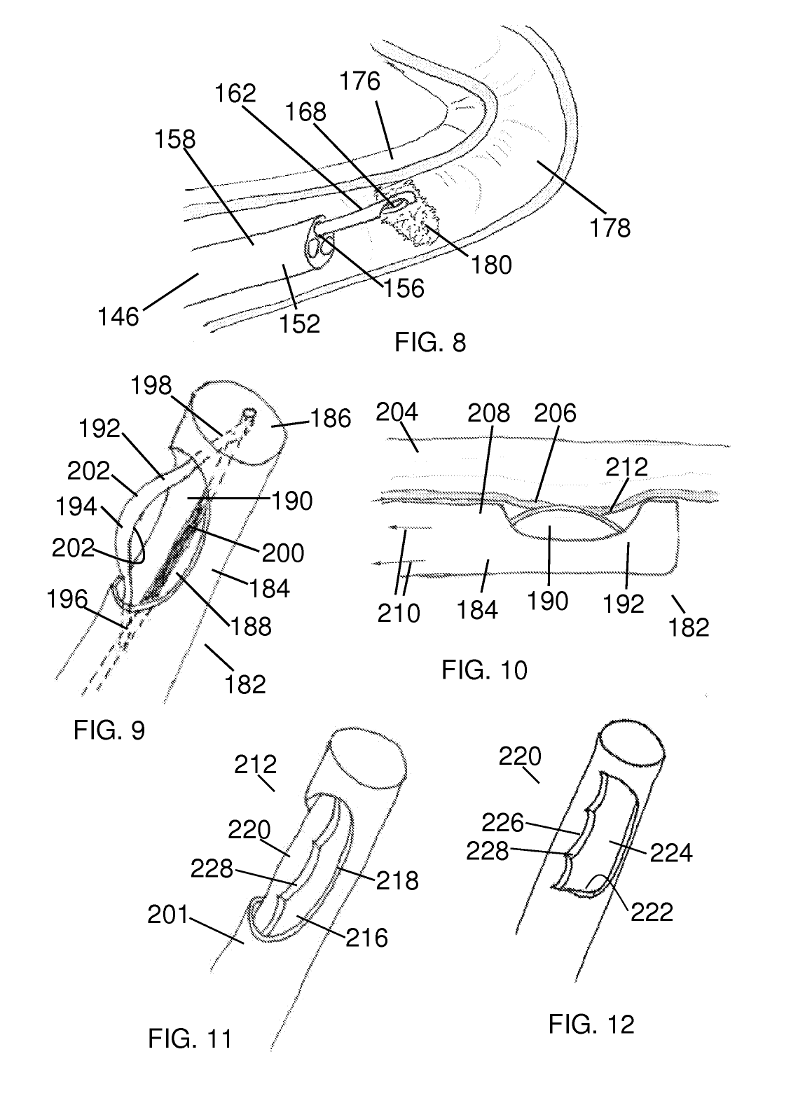

FIG. 4 generally illustrates one embodiment of an access device 146, which may be any endoscope or enteroscope known in the art or any other access devices that are commonly used to access the GI tract in minimally invasive GI tact procedures, such as colonoscopies and upper GI procedures. The access device 146 may include an elongated member 148 that may be rigid or flexible or has both rigid and flexible portions. Elongated member 148 includes a proximal end portion 150 and a distal end portion 152 which is also the distal end portion of the access device 146. The distal end portion 152 is adapted to be inserted into and through the GI tract by insertion into a natural orifice of the patient, such as the mouth, nose or anus that is in communication with the GI tract. The distal portion 152 of device 146 alternatively may be inserted through surgical incision of the of the abdomen. The distal end portion 152 of the access device 146 may be steerable so that the operator is able to maneuver and steer the access device 146 to a desired location within the GI tract. The steering mechanism may be mechanical or electrical. An actuator 154 for steering, orientating, or otherwise controlling the distal end portion 152 of the access device 146 may be located at the proximal end portion 150 of the elongated member 148. Referring to FIG. 8, access device 146 may have one or more working passageways or channels 156 extending from the proximal portion 150 of the flexible member 148 to the distal end portion 152. The passageways may be used for insertion and placement of other devices or working tools within the GI tract. Such working tools may include visualization devices, cell harvesting device, cell cutting, cell disrupting and collecting devices, tissue implantation or tissue application devices, therapeutic delivery devices, etc.

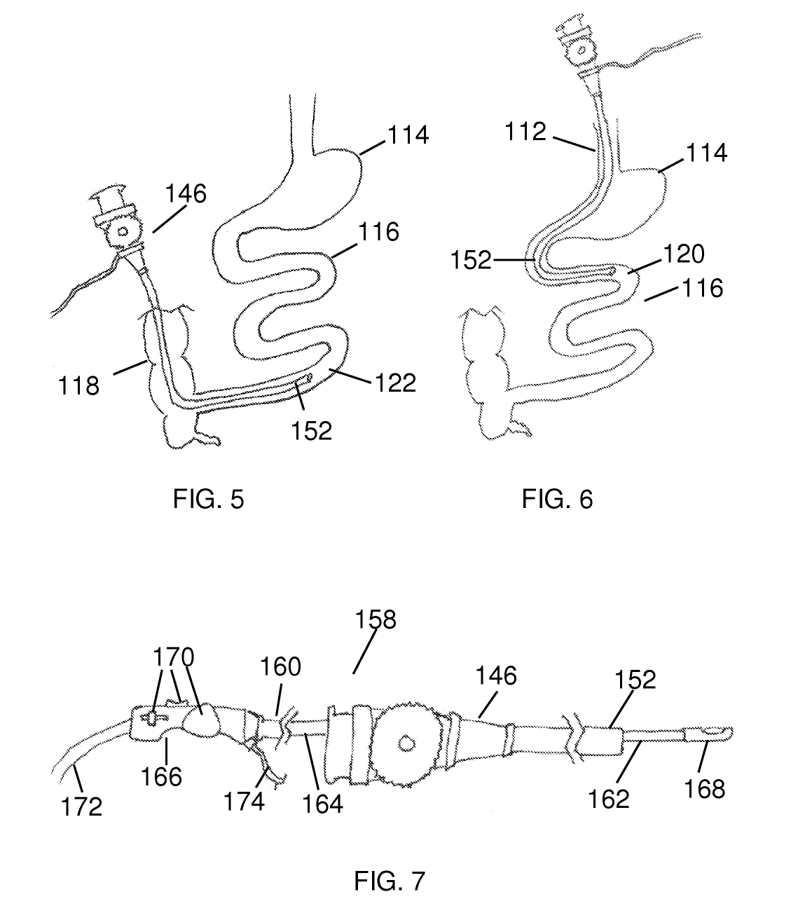

In one exemplary access technique, a transanal approach is employed to access the first or second section of the GI tract. Referring to FIG. 5, the access device 146 is inserted into the anus (not shown) and through the colon (not shown), large intestine 118 and small intestine 116 until the distal end 152 of the access device 146 reaches a desired location of the GI tract. In the illustrate embodiment, the access device 146 is inserted until it is positioned within the ileum 122.

In another exemplary access approach, a transoral approach is employed. Referring to FIG. 6, the access device 146 is inserted into the mouth (not shown) and through the esophagus 112 (partially shown), stomach 114, and small intestine 116 until the distal end 152 of the access device 146 reaches a desired location of the GI tract. In the illustrated embodiment, the access device 146 is inserted until the distal end portion 152 is positioned within the jejunum 120.

When accessing the desired section of the GI tract by any of the methods disclosed herein, the surgeon may employ a visualization device, such as a charge coupled device (CCD) or fiber optic camera, to visualize the desired section of the GI tract in which tissue is to be harvested or implanted and to ensure proper placement of the access device 146. In other embodiments, standard radiographic imaging techniques may also be used to verify proper placement of the access device 146.

When accessing the jejunum 120, a transoral approach may be preferred and when accessing the ileum 122, a transanal approach may be preferred; however, transanal and transoral approaches may be used to access the ileum, the jejunum or any other portion of the GI tract. Further, other techniques also may be employed to access a desired portion of the GI tract. Such techniques may include endoscopic abdominal approaches through incisions in the abdomen or open abdominal approaches.

Separating Cells From the GI tract

After the access device 146 has been inserted and positioned at the desired location within the GI track, further devices or tools may be inserted through the working channels 156 of the access device 146 to manipulate the cells and/or tissues of the GI tract. More particularly, cell disruption and/or collecting devices may be inserted through a working channel of the access device to harvest the cell by separating the cells from a selected portion of the GI tract and collecting/capturing the separated cells. As explained in more detail below, a cell disruption device is a device that cuts, scrapes, brushes, ablates or otherwise disrupts or separates cells from selected portions of the GI tract. A cell collecting/capturing device is a device that collects/captures the disrupted cells for removal of the cells for the selected portion of the GI tract. Cell disruption and collecting may be functions of a single device that both disrupts and collects cells. Alternatively, cell disruption may be accomplished by one or more devices, and cell collecting may be accomplished by one or more devices that are separate and distinct from the cell disruption device(s).

Referring to FIG. 7, there is shown one embodiment of a system 158 that may be used to disrupt and/or collect cells of the GI tract. The system 158 includes an access device 146, and a working device or tool 160, which may be a tissue disruption and/or collecting device.

The access device 146 includes passageway 156 (FIG. 8) that receives the working device 160 therethrough. The working device 160 is advanced through the passageway 156 so that the distal end portion 162 of working device 160 extends beyond the distal end portion 152 of the access device 146. Generally, the working device 160 includes an elongated member 164 extending from a hand piece 166. The elongated member 164 may be, for example, a flexible tube, shaft or catheter-like member that has one or more passageways (not shown) extending therethrough. In the illustrated embodiment, a cell manipulator 168 is located at the distal end portion 162 of the working device 160. The cell manipulator 168 may include any variety of cell disrupting or collecting members. Like the access device, the distal end portion 162 of the working device 160 may be steerable or guidable, so that the distal end portion 162 may be placed at selected locations and/or orientations within the GI tract.

The hand piece 166 may include various controlling and actuating mechanisms 170 that may be used to steer the distal end portion 162 of the working device 160 to the desired locations. The controlling and actuating mechanisms also may be used to actuate the cell manipulator 168. The controlling and/or actuating mechanisms 170 may be connected to the distal end portion 162 of the working device 160 and/or cell manipulator 168 by, for example, drive trains, actuating cables, electrical wires, tubes, etc. that pass through one or more passageways extending along the elongated member 164. Alternatively, the drive trains, actuating cables, electrical wires, tubes, etc may pass outside the distal end portion 152 of the access device 146

If necessary, a power supply 172 also may be connected to the working device 160 to supply power to any of the mechanisms that require electricity for operation. In the illustrated embodiment, the power supply 172 is connected to the hand piece 170. In other embodiments, the power supply 172 may be connected to other sections of the working device. In another alternative embodiment, the working device may include an internal power supply, for example, a battery. In further embodiments, the working device may be entirely mechanical for which a power supply is not required.

Furthermore, if the working device 160 includes a cell manipulator 168 that operates or requires the use of suction, a suction source 174 may be operatively connected to the working device 160. For example, when working device 160 is a tissue collection device, it may include a passageway for transporting collected cells from the distal end portion 162 of the device to the proximal end portion of the device. The suction source 174 may be in operative communication with the passageway so that the collected cells travel through the passageway under the force of suction.

FIG. 8 generally illustrates system 158, and more particularly, the distal end portion 152 of access device 146 and the distal end portion 162 of working device 160, positioned within a selected portion 176 of the GI tract. The distal end portion 162 of the working device 160 extends out of the distal end portion 152 of the access device 146, and in the illustrated embodiment out of channel 156, and the cell manipulator 168 contacts the inner wall 178 of the GI tract. In the illustrated embodiment, the cell manipulator 168 is a cell disruption element that scrapes, cuts, or otherwise disrupts cells from a selected portion 180 of the inner wall 178 of the GI tract. The selected portion 180 of the GI tract from which cells are disrupted may be, in one embodiment, the mucosal or sub-mucosal layers of GI tract wall 178.

FIGS. 9-20 illustrate multiple alterative embodiments of cell manipulators. The cell manipulators preferably, but not necessarily, include a variety of features that assist in safely disrupting and collecting selected cells from the GI tract. For example, the cell manipulators may include features that control or regulate the depth of tissue scraping or cutting. The cell manipulators may also include features that reduce the risk of puncture or laceration of the GI tract. Furthermore, the cell manipulators may be a combination of the cell disruptors and cell collectors.

Turning to FIGS. 9 and 10, the cell manipulator 182 is a cell disruptor that includes features for scraping and/or cutting cells from a selected portion of the GI tract. The cell manipulator 182 may include an outer member 184 having a closed distal end 186. The outer member 184 includes an inner cavity 188 and a side or radial opening or window 190. A cell disruption element 192 is positioned with the inner cavity 188 and aligned with opening 190. In this embodiment, the cell disruption element 192 includes a cell disruption surface such as an elongated blade or ribbon 194 having opposed ends 196 and 198 attached to a shaft 200. The blade 194 includes one or more cutting, scraping and/or shaving surfaces 202, which may be a straight or serrated edge. The shaft 200 is rotatable relative to the outer member 184 and is rotated or reciprocated to move the disruption element 190 relative to opening 190.

Referring to FIG. 10, the cell manipulator 182 may be located at a selected portion 204 of the GI tract by any of the access techniques described herein and any other suitable access techniques known in the art. The cell manipulator 182 is brought into contact with or otherwise interfaces an inner wall 206 of the GI tract. As illustrated in this figure, the disruption element 192 may be recessed a selected distance within opening 190 so that the blade 194 of the disruption element 192 is located a selected distance below the outer surface 208 of outer member 184 of the cell manipulator 182. The tissue disruption element 192 and more particularly the blade 194, may be recessed in this fashion so to limit the penetration of the disruption element 192 into the wall 206 of the GI tract. In one exemplary embodiment, the penetration is limited to a selected depth. In another embodiment, the penetration is limited to the mucosal layer. In alternative embodiments, the disruption element 192 may extend a selected distance out of the opening 190.

As shown in FIG. 10, the inner wall 206 of the GI tract enters the opening 190 of the cell manipulator 182 either by pressing the manipulator 182 against the inner wall 206 or by employing a suction mechanism that creates suction, indicated by arrows 210, which suctions a portion of the inner wall 212 into the opening 190. When suction is utilized, the cavity may be in communication with the passageway of elongated member 164 (FIG. 7) of the working device 160 which in turn is in operative communication with a suction source. When the portion of the inner wall 212 is positioned within opening 190, the shaft 200 (FIG. 9) is moved in a rotational direction relative to the outer member 184 to move the disruption element 192 relative to the outer member 184 and within the opening 190. As the disruption element 192 moves, the blade 194 cuts, scrapes, shaves or otherwise disrupts cells of the inner wall 206 to remove or separate the cells therefrom. In one embodiment, the disruption element 192 disrupts and removes cells from the mucosal layer of the inner wall. In other embodiments, the disruption element 192 disrupts and removes cells from both the mucosal and/or submucosal layers.

When a suction source is utilized with the cell manipulator 182, the disrupted cells may be suctioned into the opening 190 for collection. Furthermore, the suctioning may be used to transport the cells through a passageway within the working device 146 to the proximal end of the device. In an alternative embodiment, a separate cell collection device may be used to collect the disrupted cells. For example, a cell collection device that also uses suction to collect the cells may be employed to collect disrupted cells that have been separated or removed from inner wall 206 of the GI tract by cell manipulator 182. In alternative embodiments, the cell collection device may use other techniques to collect the cells.

FIG. 11 illustrates another embodiment of a cell manipulator 212. Similar to the previous embodiment, the cell manipulator 212 includes an outer member 214 having a closed distal end, a cavity 216 and a side opening 218. The cell manipulator also includes an inner member 220 located within the cavity 216 of the outer member 214. As shown in FIG. 12, the inner member 220 has a tube-like or cylindrical configuration. The inner member 220 includes a cavity 222 and a side opening 224 that is defined by an edge 226. The edge 226 has a cell disruption surface 228. In an alternative embodiment the inner member 220 is configured to have multiple side openings 224 arranged circumferentially around the inner member. In this embodiment there are multiple edges 226 and multiple cell disruption surfaces 228. In other embodiments, the inner member 220 may used without the outer member to disrupt cells/tissue and remove the same from the inner wall of the GI tract by placing the inner member against the inner wall of the GI tract and rotating the inner member either manually or automatically. Further, suction applied from the proximal end through the opening(s) 222 may be employed to pull the disrupted cells into opening(s) 222 and evacuate the disrupted cells from the site.

Referring back to FIG. 11, the inner member 220 is located within and coaxial with outer member 214. The inner member 220 is positioned so that cell disruption surface 228 is aligned with opening 218 of the outer member 214. Similar to the previous embodiment, in operation, the cell manipulator 212 is contacted to a selected portion of the inner wall of the GI tract so that the portion of the inner wall enters the opening 218. The inner member 220 is then rotated or reciprocated relative to the outer member 214 so that the disruption surface 228 disrupts cells of the inner wall. The disrupted cells may enter opening 224 of the inner member 220 so that such cells are collected inside of inner member 220. Alternatively, a separate cell collector may be used to collect the disrupted cells.

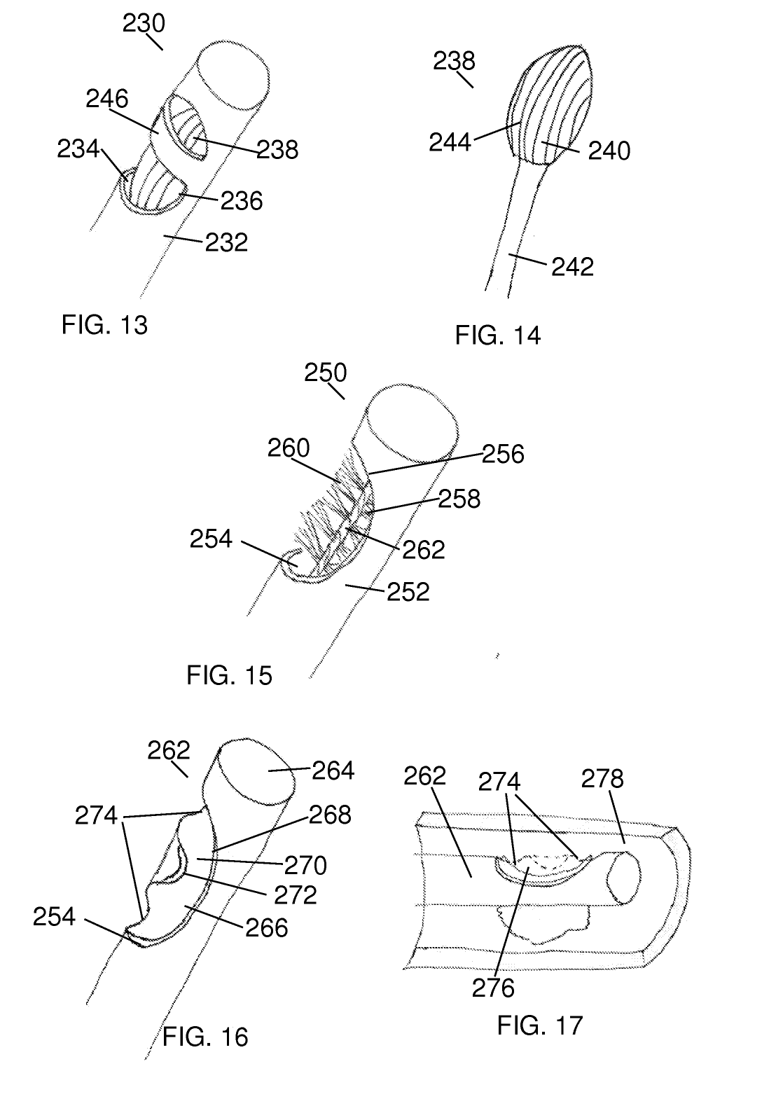

FIG. 13 illustrates yet another embodiment of a cell manipulator 230. The cell manipulator 230 includes an outer member 232 that has a closed distal end. The outer member includes a cavity 234 and a side opening 236. The cell manipulator 230 also includes an inner member 238 located within the cavity 234 of the outer member 232. In the illustrated embodiment, the inner member 238 is a cell disruptor that is a burr or has a burr-like configuration. The inner member 238 rotates within the cavity 234 of the outer member 232. Referring to FIG. 14, the inner member 238 includes a head 240 and a shaft 242. The head 240 has a plurality of cell disruption surfaces 244 extending therealong. It shall be appreciated that the cell disruption surfaces 244 may extend in any variety of directions or patterns.

In use, the cell manipulator 230 is placed against the inner wall of the GI tract so that tissue of the inner wall enters opening 236 of outer member 232. The inner member 238 is rotated or reciprocated relative to the outer member 232 so as to move the inner member relative to the opening 236. Optionally, suction also may be used with this cell manipulator 230 to suction tissue into opening 236 and/or in the collection of the disrupted cells. As the inner member 238 is rotated, the tissue disruption surfaces 244 contact and disrupt tissue. Optionally, the outer member 232 may include tissue guard 246 to limit the penetration of cell removal to a selected depth. In the illustrated embodiment in FIG. 13, the tissue guard 246 is a band or other structure that extends partially over opening 236. For example, the tissue guard 246 may be an integrated strut or panel or a separate component attached to the outer member 232 to serve as a tissue depth-limiting function.

In yet another embodiment, the tissue depth-limiting feature could be permanently or semi-permanently attached to the inner member 238, and thus rotate with said inner member. In this embodiment, an outer member as described previously may not be required.

FIG. 15 illustrates another embodiment of a cell manipulator 250. The cell manipulator 250 includes an outer member 252 that has an inner cavity 254 and a side or radial opening 256. The cell manipulator 250 also includes an inner member 258. The inner member 258 includes a plurality of cell disruption surfaces 260. In the illustrated embodiment, the cell disruption surfaces 260 are a plurality of bristles radially extending from a shaft 262. The shaft 262 may be rotated, reciprocated or vibrated relative to the outer member 252 so that the cell disruption surfaces 260 move relative to opening 256.

Similar to the other embodiments, in use, the cell manipulator 250 may be placed against a selected portion of the inner wall of the GI tract so that the cell disruption surfaces 260 contact the cells of the inner wall of the intestinal tract through opening 256. The shaft 262 of inner member 258 is rotated so that the cell disruption surfaces 260 move relative to outer member 252. As the cell disruption surfaces 260 move, they contact and disrupt the cells of the inner wall of the GI tract. Optionally, suction may also be used with this embodiment to suction the tissue into opening 256 and/or in the collection of the disrupted cells.

Referring to FIG. 16, cell manipulator 262 includes a closed distal end portion 264 and an inner cavity 266. The cell manipulator 262 also includes an edge 268 that forms an opening 270 in communication with the inner cavity 266. The edge 268 includes a dissection portion 272 extending between two cell disruption portions or cutting blades 274. The dissection portion 272 is located between and extends out from or beyond the cutting blades 274, which cutting blades have a concave shape in the embodiment shown.

As shown in FIG. 17, the cell manipulator 262 may be used to remove a layer of cells or tissue 276 from the inner wall 278 of the GI tract. In one embodiment, cell manipulator 262 may be used to remove a portion of the mucosal layer of the inner wall 278 of the GI tract. In use, the dissection portion 272 pierces and is inserted a selected depth into the inner wall 278 of the GI tract. The cell manipulator 262 is then rotated, reciprocated or vibrated so that the cutting blades 274 contact and cut a layer of cells 276. As the cell manipulator 262 rotates, the dissection portion lifts or separates the cells 276 from the inner wall 278 of the GI tract.

Once a desired amount of cells have been cut, the cells or layer of cells/tissue 276 are removed from the wall of the GI tract. In one embodiment, after the desired layer of cells/tissue has been initially cut and a portion of the layer is still attached to the inner wall, the layer 276 may be removed or cut from the inner wall of the GI tract by retracting the cell manipulator and severing the attached portion of the layer with cutting blades 274. The layer then may be collected in the inner cavity 266. In an alternative embodiment, the cell manipulator 262 may include an element, such as a mandrel, in which the layer of cells is rolled around for removal and transfer to a transplant site. The ability to removed layer of cells may be desired in procedures that include transplanting or grafting relatively larger portions of intact cells from one section of the GI tract to another section of the tract.

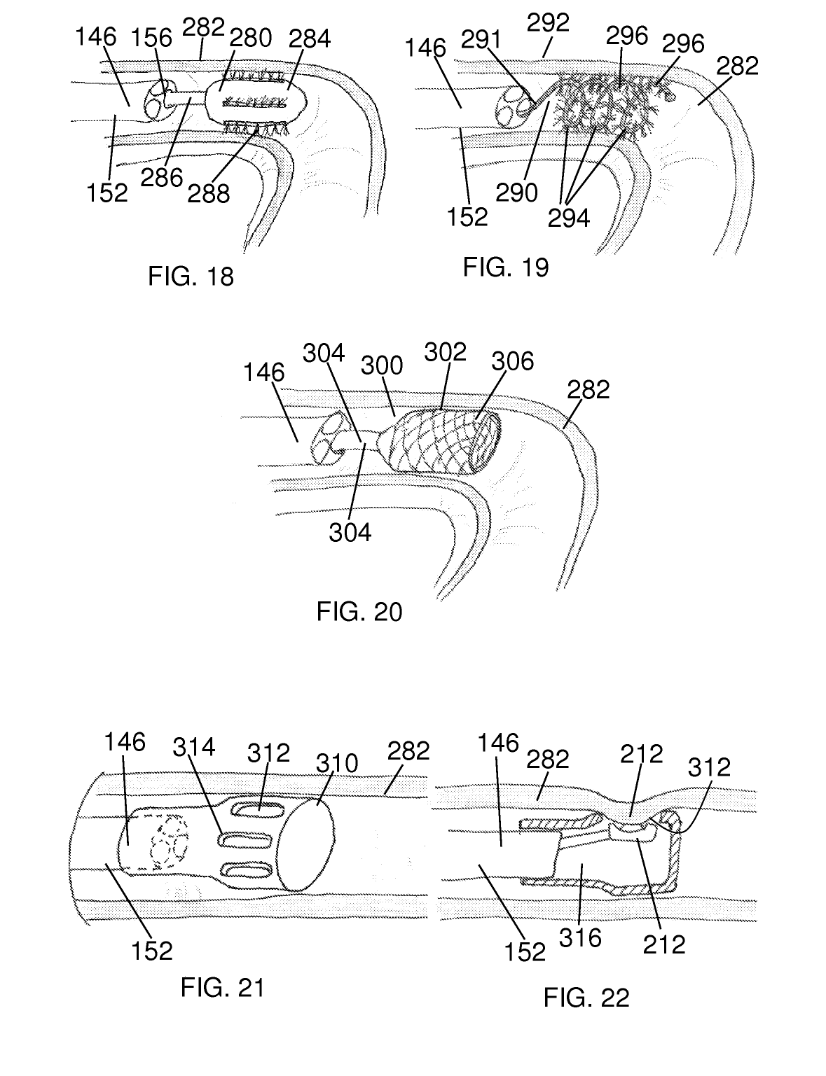

FIG. 18 illustrates another embodiment of a cell manipulator 280 shown positioned within a portion of the GI tract 282. In this embodiment, the cell manipulator 280 includes an expandable structure 284, which is some embodiments may be an inflatable structure. The expandable structure may be, for example, a balloon. The expandable structure also may be non-inflatable structures which have expandable geometries that do not require inflation for expansion. The expandable structure 284 is located at the distal end of a shaft 286. When the expandable structure is an inflatable structure, the shaft 286 may also be a fluid delivery member which delivers fluid, such as any suitable gas or liquid, into the expandable structure 284 to expand the structure. The expandable structure 284 may be made from a flexible or stretchable material that stretches to expand the structure. Alternatively, the expandable structure 284 may be made from a non-stretchable material that prevents the structure from expanding beyond a selected size. Furthermore, the expandable structure 284 may include constraints, such as tethers or internal baffles, that constrain the expansion of the structure 284.

The expandable structure 284 includes cell disruption elements 288 located on an exterior surface of the expandable structure 284. In the illustrated embodiment, the disruption elements 288 are a plurality of bristles that include tissue disruption surfaces which contact and disrupt tissue. The bristles can be metallic or synthetic polymer fiber that has a stiffness sufficient to disrupt cells of the inner wall of the GI tract. In one embodiment, the bristles have a stiffness that allows disruption of the mucosal layer but are not stiff enough to penetrate other layers of the inner wall of the GI tract. In other embodiments, the cell disruption elements 228 may be elongated rigid members (like blades), or wire/ribbon filaments attached lengthwise to the exterior of the expandable structure 284 such that the ends of the filaments are anchored at either end of the expandable structure. In one embodiment, the middle portion of the filaments is unattached such that the middle portions may be spaced from or floats off of the surface of the expandable structure. The expandable structure shown in FIG. 18 depicts cell disruption elements mounted in an axial direction on the expandable member. It is understood, however that the cell disruption elements may be mounted in any direction, for example, the cell disruption elements may be mounted in a circumferential fashion.

When in use, the distal end portion 152 of the access device 146 is positioned at a desired location within the GI tract 282. The expandable structure 284, in an unexpanded state, is feed through a passageway 156 of the access device 146 and beyond the distal end portion 152 of the access device 146. The expandable structure 284 then is expanded to a selected size. In one exemplary embodiment, the expandable structure 284 is expanded to a size that approximates the size of the lumen of the particular section of the GI tract. When the expandable structure 284 is an inflatable device, the expansion size and pressure within the expandable structure 284 can be controlled. Controlling the size of the expandable structure assists the user in controlling the depth at which the disruption elements 288 penetrate the inner wall of the GI tract 282. In other words, the expansion size may be selected so that the disruption elements 288 disrupt cells only up to a selected depth of the inner wall of the GI tract.

Once expanded, the expandable structure 284 may be rotated either manually or automatically. Optionally or alternatively, the expandable structure may be moved back and forth in an axial direction. Such back and forth movement may be particularly appropriate when the cell disruption elements are mounted circumferentially on the expandable structure. The movement of the expandable structure 284 may be controlled by moving the shaft 286 in the desired axial and/or rotational directions. As the expanded structure 284 is moved, the disruption elements 288 contact to the inner wall of the GI tract 282 to disrupt the cells. The disrupted cells are collected, contained or otherwise remain associated with the disruption elements 288.

Once the desired amount of cells is collected, the expandable structure 284 is returned to its unexpanded state and withdrawn through the passageway 156 of the access device 146 with the disrupted cells maintained in association with the disruption elements 288. The expandable structure 284 may be withdrawn through the passageway 156 by retraction of shaft 286. The disrupted cells may then be collected from the cell disruption elements 288.

FIG. 19 illustrates another embodiment of a cell manipulator 290 positioned within a portion of the GI tract 282. The cell manipulator 290 includes an elongated member 291 having a distal end portion 292 that includes a predetermined or selected configuration. In the illustrate embodiment, the distal end portion 292 of the cell manipulator 290 includes a plurality of loops or windings 294. The elongated member 291 may be, for example, a wire or ribbon. In one embodiment, the elongated member 291 is comprised of a metal or metal alloy. The elongated member 291 also may be comprised of a shape memory alloy, such as Nitinol.

The distal end portion 292 includes a plurality of cell disruption elements 296 extending therefrom. In the illustrated embodiment, the disruption elements are bristles embedded in the wire. In the embodiment shown, the elongated member 291 is a twisted wire in which the bristles are twisted within the wire. The bristles include cell disruption surfaces which contact and disrupt cells.

In use, the distal end portion 152 of the access device 146 is placed within the GI tract 282 and the cell manipulator 290 is inserted through the passageway 156 and beyond the distal end portion 152 of the access device. When the cells manipulator 290 is constructed from a shape memory material, the distal end portion 292 may be inserted through the passageway 156 in a substantially straight configuration. As the distal end portion 292 of the cell manipulator 290 extends beyond the distal end portion 156 of the access device, the distal end portion 292 of the cell manipulator 290 is allowed to return to its predetermined configuration, allowing the cell disruption elements 296 to contact the inner wall of the GI tract 282. When the predetermined shape of the distal end portion 292 of cell manipulator includes coils, the diameter of the coils and the thickness of the wire may determine the force with which the disruption elements 296 contact the inner wall of the GI tract 282, thus providing a predetermined or selected penetration depth. Once deployed, the distal end portion 292 of the cell manipulator 290 may be rotated and moved back-and-forth to disrupt and collect the cells. After the desired amount of cells has been disrupted, the cell manipulator 290 may be retracted back through the access device 146 with the cells maintained in the disruption elements 296.

FIG. 20 illustrates another embodiment of a cell manipulator 300 positioned within a portion of the GI tract 282. The cell manipulator 300 includes an expandable structure 302 located at the distal end of a shaft 304. In this embodiment, the expandable structure 302 is an expandable cage having a network of interconnected ligaments 306, which include cell disruption surfaces.

The expandable structure 302 may be made from a metal or polymer material. Furthermore, the expandable structure 302 may be made from a shape memory material, such as a metal or polymer, that can transform from an unexpanded or condensed configuration to a larger expanded configuration. In an exemplary embodiment, the expandable structure 302 has a deployment configuration for deployment into the GI tract and then a deployed configuration within the GI tract. For example, the expandable structure 302 may be made of a metal wire and constructed in a stent-like manner so that it can have a deployment configuration in which the structure 302 has a size and shape that allows the structure 302 to be advanced through a passageway 156 of an access device 146. As or after the structure 302 is advanced from the passageway 156 of the access device 146, the structure expands to a larger deployed configuration. When the expandable structure 302 is made of a shape memory material, such as Nitinol, the super elastic properties of the material cause the structure 302 to transform from the unexpanded configuration to the expanded configuration.

Once expanded, the expandable structure 302 is contacted to the inner wall of the GI tract 282 and rotated, moved back and forth or vibrated to disrupt cells and remove them from inner wall. The wire of the cage may be configured so as to have cell disruption surfaces in the form of sharpened edges to facilitate tissue removal.

In a further embodiment, a cell manipulator (not shown) may include a cage construct similar to the expanded configuration of expandable structure 302. This cage construct may be constructed so that it is sufficiently flexible and can be slid over the outer surface of the access device to a selected location within the GI tract.

In any of the above described embodiments of cell manipulators, the systems and mechanisms employed to disrupt cells within the GI tract may include a tissue guard that may protect surrounding tissue during cell removal and collection and restrict or limit the penetration depth of the cell disruptors.

In one embodiment, the tissue guard may be a cage-like structure similar to the expandable structure 302 illustrated in FIG. 20. The cage-like structure includes a plurality of interconnected ligaments that define a plurality of openings. In this embodiment, the cage like structure may be used with any of the cell manipulators discussed above. The cell manipulator including, for example, a blade, burr, bristles, etc. may be deployed inside of the cage. In use, the cage is pressed against the inner wall of GI tract so that portions of the inner wall extend through the openings of the cage. The cell manipulator may then be utilized to disrupt the tissue portions extending through the openings. In such an embodiment, the thickness of the ligaments that define the cage would act as a control measure to prevent the cutter from cutting too deep into the tissue.

FIGS. 21 and 22 illustrate another embodiment of a tissue guard 310. In this embodiment the tissue guard 310 is a cap that may be located on the distal end portion 152 of the access device 146. The tissue guard 310 may be similar to those that are sometimes used in endoscopic mucosal resection procedures. The tissue guard 310 may be open or closed ended. Furthermore, the tissue guard 310 may be made of any suitable material, such as a metal or polymer. In one embodiment, the polymer is a clear polymer to allow visual inspection. The tissue guard 310 may be integral with the distal end portion 152 of the access device 146 or it may be attached by friction fit or an adhesive.

In the embodiment shown, the tissue guard 310 is a closed-end cap having a generally cylindrically shaped outer wall 314 defining an inner cavity or space 316 (FIG. 22). The guard 310 also includes a plurality of elongated fenestrations, openings or slots 312 spaced around its circumference and extending in a longitudinal or axial aspect. In an alternative embodiment the cap or end-mounted tissue guard 310a may have fenestration(s) 312a that extend in a circumferential aspect as illustrated in FIG. 32. In the embodiment shown in FIG. 22, the guard 310 has a larger outer diameter than the access device 146, and thus, the guard 310 readily contacts the inner wall of the GI tract 282. In other embodiments, the guard 310 may have a smaller diameter. In use, the tissue guard 310 is pressed against the inner wall so that portions 212 of the inner wall protrude through the fenestrations 312. When a closed-end guard is employed, suction may be used to pull portions 212 of the inner wall of the GI tract through the fenestrations 312.

With the portions of the inner wall protruding 212 through the fenestrations 312 of the tissue guard 310, any of the above described cell manipulators may be deployed through the access device 176 as described above and into the interior 316 of the guard 310. The cell manipulators 168 may contact a portion of the tissue that is exposed through the fenestrations, as shown in FIG. 22, to disrupt cells and remove cells from the inner wall. The thickness of the guard 310 and the suction force, when used, may be varied to control the tissue cut depth. Thus, the tissue guard 310 may assist in controlling depth of cut.

In other embodiments, the tissue guard 310 may be a tissue manipulator that includes a tissue disruption element. As shown in FIG. 32 tissue guard 310 includes tissue disruption surfaces in the form of sharp fenestration edges 311 that may be used to disrupt cells alone, as shown in FIG. 33, or may be used in combination with another cell manipulator. In the embodiment shown in FIG. 33, when tissue protrudes through the fenestration(s) via manual pressure or suction, the cutting/scraping edge 311 of the fenestration contacts the tissue. By moving the cap or end-mounted tissue guard 310 in the axial direction, tissue layers 313 may be shaved/cut/scraped or otherwise disrupted and subsequently suctioned into the fenestrations and through the device. Alternatively, if the fenestration(s) are arranged circumferentially as in FIG. 30 and further configured with tissue disruption surfaces such as sharp edges so as to disrupt the tissue, then tissue disruption may be achieved by rotating the tissue guard/cap about its axis.

Cell Collection Cells/Tissue

The disrupted cells may be collected by any suitable method or device. Such devices may employ, for example, suction force, cell adhesion, cell entanglement, forceps-like devices and/or collection containers. As discussed above, in one embodiment, suction may be used to evacuate the separated tissue/cells through a passageway located in the working device. The separated tissue/cells may be collected by applying a suction force to pull the tissue/cells into the device and suction the tissue/cells through the device into an external collection chamber that is operatively connected to the suction source.

In an alternative embodiment, the tissue/cells may be collected and retained within an internal chamber of the working device and retrieved when the working device is withdrawn from the access device. In a further embodiment and as discussed above with respect to device that employ bristles, the disrupted tissue/cells may be become entangled and retained within the bristles and removed and collected when the bristles are withdrawn from the access device.

Cell Processing

Once the tissue/cells have been collected they may be subjected to cell processing, if desired. Typically, the collected tissue/cells are removed from the GI tract to undergo cell processing: however, cell processing also may take place within the GI tract.

The cells may be subjected to separation processes to separate selected cells from other tissue or cells, washing procedures to remove blood, fat or other cells, concentrating procedures and/or other cell processing procedures. The cells may, as desired, be suspended in a preservative or other solution for processing storage and/or subsequent implanting. The cells also may be attached to a support matrix or support surface for later implantation or for assisting in keeping the cells intact during implantation. Furthermore, the cells also may be subjected to culturing for increasing cell population or enhancing the viability or promoting implantation. The cells also may be placed in a fluid to create a suspension of cells which may be utilized in the re-implantation of the cells. The cells also may be subjected to a combination of processing procedures. For example, the cells may be subjected to a cell separation process to separate selected cells from other cells, and the selected cells may then be subjected to culturing to increase the cell population.

Such cell processing may employ any suitable techniques and/or instruments known in the art. For example, the cells may be subjected to the processes described in U.S. Patent Application Pub. No. 2008/0014181, filed Apr. 23, 2007 and to published Jan. 17, 2008, U.S. Pat. No. 6,316,247, filed Jun. 15, 1999 and issued Nov. 13, 2001, U.S. Pat. No. 5,786,207, filed May 28, 1997 and issued Jul. 28, 1998, and U.S. Pat. No. 5,372,945, filed Jul. 31, 1992 and issued Dec. 13, 1994, all of which are hereby incorporated herein by reference. Such processing techniques may include the use of any suitable instruments, such as cell separation devices and centrifuges.

In one embodiment, if the tissue is in large pieces or layers, the cells may be scraped from the tissue and placed into a container. Fluids may be added to the container to interact with the cells and aid in isolating and washing desired cells, such as the L-Cells containing the GLP-1 and Peptide -YY hormones. Once isolated, these cells may be activated and placed in a fluid suspension. This suspension may contain fluid that acts to enhance the ability of the suspension (with L-Cells) to adhere to the new surface onto which it is eventually placed.

Re-implantation of Cells

After the cells have been collected from a first section of the GI tract, and optionally processed, the cells may be re-implanted into a second section of the GI tract. The cells may be re-implanted using any procedures and techniques know in the art.

The second section of the GI tract may be accessed by using the devices and techniques disclosed herein or any suitable devices and technique known in the art. For example, the second section of the GI tract may be accessed by inserting access device 146 (FIG. 4) into the GI tract through a transoral or transanal approach as shown in FIGS. 5 and 6.

Optionally, prior to re-implantation of the collected cells, the second section of the GI tract may be prepare or treated to receive implantation of the cells. The preparation procedures of the second section of GI tract may include removing some or most of the mucosal tissue layer of the second section and/or applying therapeutic agents to the second section. Removal of the mucosa tissue may be accomplished using the same or similar instruments and techniques described above with respect to the cell disrupting procedures and cell manipulators. For example, any of the above-described cell manipulators shown in FIGS. 9-20 may be used to remove the mucosal layer or portion of the mucosal layer from the second section of the GI tract. The mucosal layer in this area of the second section is removed so that the newly applied cells may proliferate with the healing process. It is believed that removal of the mucosa of the second area will promote the newly collected cells to multiply and regrow during the healing process, thus replacing the old mucosal layer.

Optionally, as the cells of the mucosal layer are removed during preparation of the second section of the GI tract, such removed cells may be collected for re-implantation in the first section or another section of the GI tract. It is believed that this could potentially help with obesity by changing the metabolic processes and creating satiety of the first section or other sections.

After the second section of the GI tract has been accessed, and optionally prepared for re-implantation of the cells, the cells may then be deposited on the inner wall of the second section using any suitable techniques and instruments described herein or know in the art. Implantation of the cells creates a new mucosal layer on second section that is generated from the cells collected from the first section. When the first section is the ileum and the second section is the jejunum, the mucosal layer formed on the jejunum from the transplanted L-cell of the ileum will be rich in GLP-1 and peptide-YY hormones thus, providing better glucose homeostasis and mediating the diabetic state.

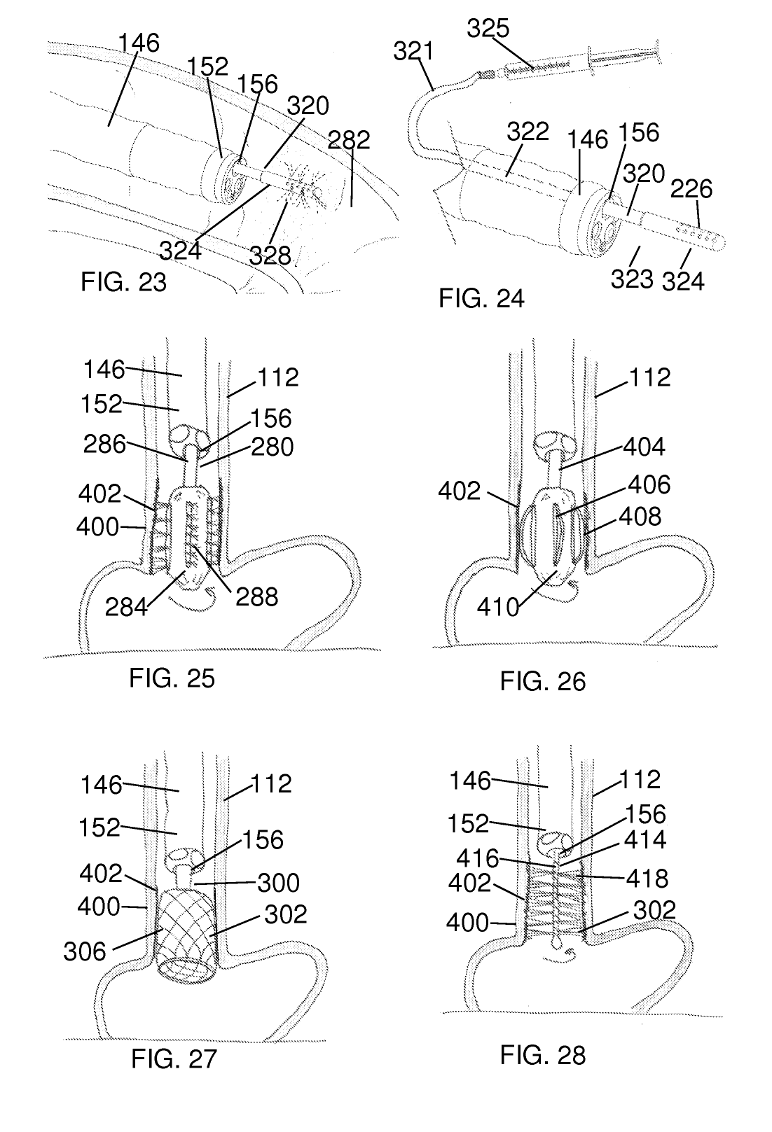

FIGS. 23 and 24 illustrate one example of a method and device for applying the collected cells to a section of the GI tract. When the cells have been processed or placed in suspension, the cell suspension fluid may be sprayed onto the inner wall of a section of the GI tract, as generally shown in FIG. 23.

Turning to FIG. 24, there is shown an application device 320 inserted through a passageway 156 of access device 146. The application device 320 may include an elongated fluid conduit 322. The proximal end 321 of the fluid conduit may terminate at a fluid injection device 325 such as a syringe or automated pump mechanism which forces the cell suspension fluid through the conduit. The distal end 323 includes an applicator 324. In one embodiment, the fluid conduit 322 of the application device 320 is flexible and may be positionally controlled from the proximal end by an actuators and control mechanisms.

In the illustrated embodiment, the applicator 324 includes a plurality of spray openings 326 for ejecting a spray 328 of the cell suspension fluid. The spray openings 326 may be located around the entire circumference of the applicator 324 to produce a spray pattern in all directions or may be located in selected sections of the applicator 324 to produce a directional spray pattern. Alternatively, a single spray nozzle may be used to gain accuracy of placement of the cell suspension.

Referring back to FIG. 23, the distal end portion 152 of the access device 146 is placed at a selection section of the GI tract 282. The application device 320 is then inserted through passageway 156 and advanced beyond the distal end 152. The cell suspension then may be transferred through the fluid conduit 322 and sprayed out through the openings 326 of the applicator 324. The spray 328 applies the cells to the inner wall of GI tract where the cells will grow and proliferate. When the mucosal layer of the inner wall has been removed, even partially, the transplanted cells attach to the second section and multiply and regrow during the healing process.

An alternative embodiment of a cell application device is shown in FIG. 54. In this embodiment, a hypodermic needle 330 is positioned at the distal end of the application device 320. This allows for direct injection of the cell suspension fluid into a specific site 331 in the GI wall.

FIG. 55 shows a cap-and-needle type embodiment of a cell suspension fluid applicator. A cap 332 is shown mounted to the end of an access device 146 such as an endoscope. As with cap cell manipulator embodiments disclosed in this application, the cap preferably has a soft, elastic or flexible proximal portion 333 which fits tightly over the end of the access device 146. A more rigid distal portion 334 is substantially open and forms a suction chamber 335. A needle 330 may be retractably disposed within the suction chamber. A suction opening is in communication with the chamber and is also in communication with the suction conduit of the access device 146. When suction is applied via the access device, tissue 336 from the GI wall is pulled into the suction chamber as shown in FIG. 56. The needle 330 is then advanced into the tissue and the cell suspension fluid 337 may be injected. It should be understood that the cap 332 may be configured any number of ways while still offering the same basic functionality, such as with differently sized or shaped suction chambers. Multiple needles and tubes may be utilized of varying gauges and lengths.

In other embodiments, when the cells have been collected as a layer, group or clump and are to be re-implanted as complete or partial tissue layers, the cells may be placed in an application device that transports and places the cells as the complete or partial tissue layers in the second section of the GI tract. In one embodiment, the cells layers may be transferred and applied by an application device that maintains the layer of cells in a compacted or rolled-up state until the cell layer is applied to the section of GI tract. If the tissue is transplanted in this manner, it may be necessary to utilize sutures or some other faster technologies, such as tissue adhesive, stitches or staples to hold the grafted tissue layer in place during the healing process.

In one example, the layer of cells could be collected from a first section of the GI tract, by a tissue disruption element as described above, and placed into a containment vessel at or near the end of the working device. The working device along with the containment vessel could then be located in a second section of the GI tract and the containment vessel could release or apply the tissue to the second section of the GI tract for implantation therein. In yet another embodiment, the transplanted cells are placed inside a free-floating (or tethered) delivery device such as a water-soluble capsule which is swallowed by the patient. The capsule wall composition designed with a time-release formula that allows the capsule to travel for a predetermined time down the GI tract where it ruptures to release the transplanted cells.

Treatment of Barrett's Esophagus

Also disclosed herein are methods for treating Barrett's disease and other esophageal related diseases. The methods generally include cutting scraping or otherwise disrupting esophageal tissue to remove abnormal or diseased cells of the esophagus. More particularly, the methods may include removing a thin layer of cell/tissue that may be 1 to a few cells thick or a layer having a thickness between about 50 microns and about 500 microns. In other embodiments, the thickness may be at least 50 microns thick or at most 500 microns thick. The method may also include removing tissue layers of greater thicknesses. Furthermore, the methods may include one or more devices that remove cells/tissue around the full circumference of the inner wall of the esophagus. Alternatively, the method may include removing cell/tissue from partially around the circumference of the inner wall of the esophagus.

The methods disclosed herein for disrupting and collecting cells from the esophagus and other portions of the GI tract also may be used to disrupt and collect cells for the purpose conducing cell examination, such as histopathological examination. For example, the methods of disrupting and colleting cells from the GI tract may be used to perform biopsies of cells/tissue.

The method of treating the esophagus, and more particularly Barrett's, may utilize any of the cell manipulation devices disclosed herein. For instance, the methods for treating and/or removing cells from the esophagus may utilize any of the cell manipulators described in FIGS. 9-20.

Turning now to FIG. 25, cell manipulator 280, which has been previously describe with respect to FIG. 18, is shown positioned within the esophagus 112 of the GI tract. When in use, the distal end portion 152 of the access device 146 is positioned at a desired location within the esophagus 112. The expandable structure 284, in an unexpanded state, is feed through a passageway 156 of the access device 146 and beyond the distal end portion 152 of the access device 146. The expandable structure 284 is then expanded to a selected size. In the expanded configuration, the cell disruption elements 288 contact a selected portion 400 of the esophagus which includes cells/tissue 402 to be disrupted or separated and removed form the esophagus. In one embodiment, cells 402 are cells associated or afflicted with Barrett's.

The expandable structure 284 may be rotated, reciprocated or vibrated either manually or automatically. Optionally or alternatively, the expandable structure 284 may be moved back and/or forth in an axial direction. The movement of the expandable structure 284 may be controlled by moving the shaft 286 in the desired axial and/or rotational directions. As the expanded structure 284 is moved, the disruption elements 288 contact to the selected cells 402 and disrupt and remove the cells from the esophagus. When the extendable structure 284 is rotated, the cell manipulator 280 disrupts tissue from the entire circumference of the selected portion 400 of the esophagus 112.

Once the desired amount of cells is removed from inner wall of the esophagus 112, the expandable structure 284 is returned to its unexpanded state and withdrawn through the passageway 156. The expandable structure 284 may be withdrawn through the passageways 156 by retraction of shaft 286. The disrupted cells may then be collected or collected from the cell disruption elements 288 and tested.

FIG. 26 illustrates another embodiment of a cell manipulator 404 that may be used to disrupt tissue of the GI tract, such as in esophagus 112. The cell manipulator 404 is similar to that of cell manipulator 280, except that the cell disruption elements 406 are a plurality of axially extending blades attached to the expandable structure 410, which may be a wire or ribbon. The blades include tissue disruption surfaces 408 which cut, scrape or otherwise disrupt tissue.

In FIG. 27, cell manipulator 300, which was previous describe with respect to FIG. 20, is shown positioned within the esophagus 112. The cell manipulator includes an expandable structure 302. The expandable structure 302, in an unexpanded configuration, is inserted through the passageway 156 of the access device 146 and advanced beyond the distal end portion 152 of the access device 146. As or after the structure 302 is advanced from the passageway 156 of the access device 146, the structure 302 expands to a larger deployed configuration. When the expandable structure 302 is made of a shape memory material, such as Nitinol, the super elastic properties of the material cause the structure 302 to transform from the unexpanded configuration to the expanded configuration.

Once expanded, the expandable structure 302 is contacted to a selected section 400 of the esophagus 112 and rotated, vibrated and/or moved back and forth to disrupt cells 402 and remove them from the esophagus 112. After the desired amount of cells 402 have been removed, the expanded structure 302 may be retracted back into passageway 156 wherein it assumes it unexpanded configuration for retraction through the passageway 156.

In a further embodiment, the cell manipulator may include cage-like construct similar to the expanded configuration of expandable structure 302. This cage-like construct may be constructed so that it is sufficiently flexible and can be slid over the outer surface of the access device to a selected location within the within the esophagus. Once in the desired position, the cage-like structure may be placed in contact with tissue and rotated and/or moved back and forth to disrupt and remove selected cells from the esophagus.

Referring now to FIG. 28, a cell manipulator 414 is shown positioned within the esophagus. The cell manipulator includes a shaft 416 having cell disruption elements 418 extending radially therefrom. In the illustrated embodiment, the cell disruption elements 418 are bristles that extend radially from the shaft 416. In one embodiment, the bristles are sized so as to contact the entire circumference of the selected section 400 the esophagus 112 when the bristles are positioned therein. The shaft 416 may be a twisted wire wherein the bristles are mounted to the shaft 416 by being twisted within the wire. Furthermore, the bristles may be configured so that they fold against the shaft 416 for insertion and retraction into passageway 156 of the access device 146.

In use, the cell manipulator 414 is inserted into passageway 156 of access device 146 and the distal end portion of the cell manipulator 414 is extended beyond the distal end portion 152 of the access device. The cell disruption elements 418 are placed in contact with selected cells 402 to be removed. The cell manipulator 414 is then rotated, reciprocated or vibrated so that the cell disruption elements 418 contact and remove the selected cells 402. Once the selected cells are disrupted and removed from the esophagus 112, the cell manipulator is retracted back into passageway 156 of the access device 146.

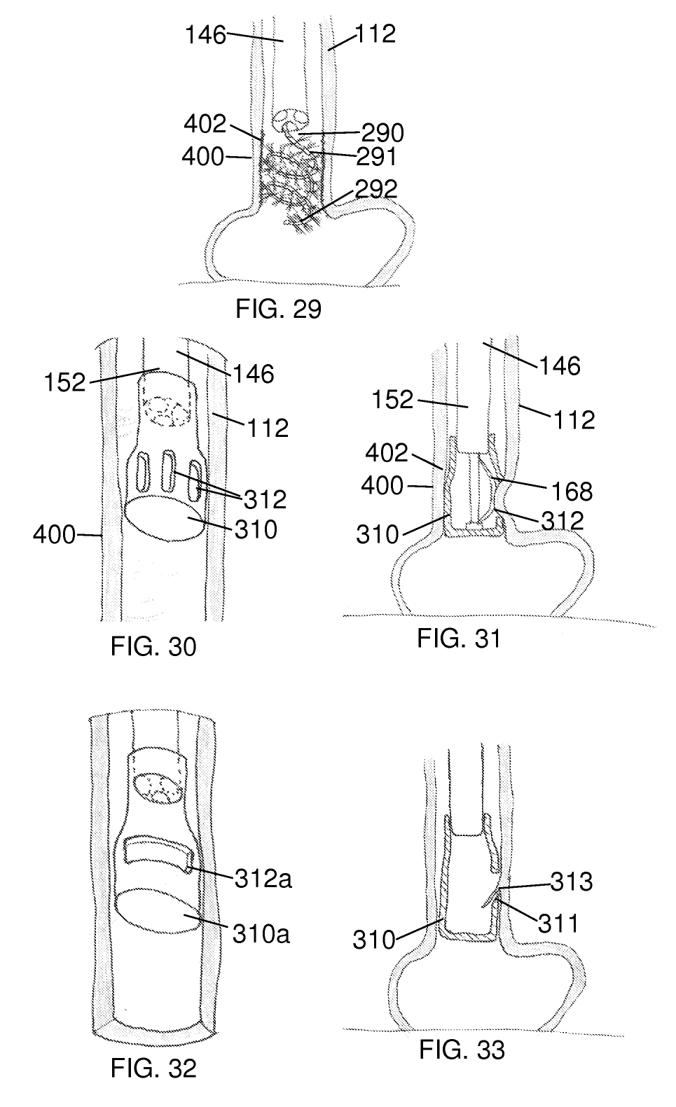

In FIG. 29, cell manipulator 290, which was previously described with respect to FIG. 19, is shown positioned within the esophagus 112. The cell manipulator 290 includes an elongated member 291 having a distal end portion 292 that includes a predetermined or selected configuration. The distal end portion also includes cell disruption elements 296 that are adapted to contact and disrupt tissue. In the illustrated embodiment, the disruption elements 296 are bristles.

In use, the distal end portion 152 of the access device 146 is placed within esophagus 112 and the cell manipulator 290 is inserted through the passageway 156 and beyond the distal end portion 152 of the access device. When the cell manipulator 290 is constructed from a shape memory material, the distal end portion 292 may be inserted through the passageway 156 in a substantially straight configuration. As the distal end portion 292 of the cell manipulator 290 extends beyond the distal end portion 156 of the access device, the distal end portion 292 of the cell manipulator 290 is allowed to return to its predetermined configuration, allowing the cell disruption elements 296 to contact the inner wall of the GI tract 282. When the predetermined shape of the distal end portion 292 of cell manipulator includes coils, the diameter of the coils and the thickness of the wire may determine the force with which the disruption elements 296 contact the inner wall of the esophagus 112, thus providing a predetermined or selected penetration depth. Once deployed, the distal end portion 292 of the cell manipulator 290 may be rotated, pushed back-and-forth, reciprocated or vibrated to disrupt selected cells within the esophagus. After the desired amount of cells has been disrupted, the cell manipulator 290 may be retracted back through the access device 146.

When the cell manipulators described herein are utilized to disrupt and remove cells/tissue from the esophagus, such manipulators may be used in conjunction with tissue guards that may protect adjacent tissue and limit the depth and penetration of the cell disruption elements. FIGS. 30 and 31 illustrate the use of tissue guard 310, which has been described with respect to FIGS. 21 and 22, in a procedure for removing selected cells from the esophagus 112. As described above, the tissue guard 310 may be a cap that is located on the distal end portion 156 of the access device 146. The guard 310 includes a plurality of elongated fenestrations or slots 312 spaced around its circumference. The tissue guard 310 is pressed against the inner wall so that portions of the inner wall protrude through the fenestrations 312. When a closed-end guard is employed, suction may be used to pull portions of the inner wall of the esophagus through the fenestrations 312.

With the portions of the esophagus protruding through the fenestrations 112, any of the cell manipulators described herein may be deployed through the access device 146 as described above and into the interior of the guard 310. In the illustrated embodiment, cell manipulator 168 contacts the tissue protruding through fenestration 112, as shown in FIG. 31, to disrupt cells and remove cells from the inner wall of the esophagus.

In other embodiments, the tissue guard 310 may be a tissue manipulator that includes a tissue disruption element. As shown in FIG. 32 tissue guard 310 includes tissue disruption surfaces in the form of sharp fenestration edges 311 that may be used to disrupt cells alone, as shown in FIG. 33, or may be used in combination with another cell manipulator. In the embodiment shown in FIG. 33, when tissue of the esophagus protrudes through the fenestration(s) via manual pressure or suction, the cutting/scraping edge 311 of the fenestration contacts the tissue. By moving the cap or end-mounted tissue guard 310 in the axial direction, tissue layers 313 of the esophagus may be shaved/cut/scraped or otherwise disrupted and subsequently suctioned into the fenestrations and through the device. Alternatively, if the fenestration(s) may be arranged circumferentially as in FIG. 30 and further configured with sharp edges so as to disrupt the tissue, then tissue disruption may be achieved by rotating the tissue guard/cap about its axis. In the case of either axial or circumferential fenestrations, additional components such as cutting blades made of metal, ceramic or other hard cutting material, may be incorporated into the fenestrations so as to better facilitate removal of the tissue/cells.

In an alternative embodiment, a cage or screen-like structure may also be used as a tissue guard. In this embodiment, the cage or screen-like structure is positioned against the inner wall of the esophagus so that the selected tissue portions of the esophagus extend or protrude through the opening of the cage or screen-like structure. A cell manipulator is deployed within the cage or screen-like structure and is used to disrupt cells from the tissue protruding through the openings of the cage or screen-like structure.

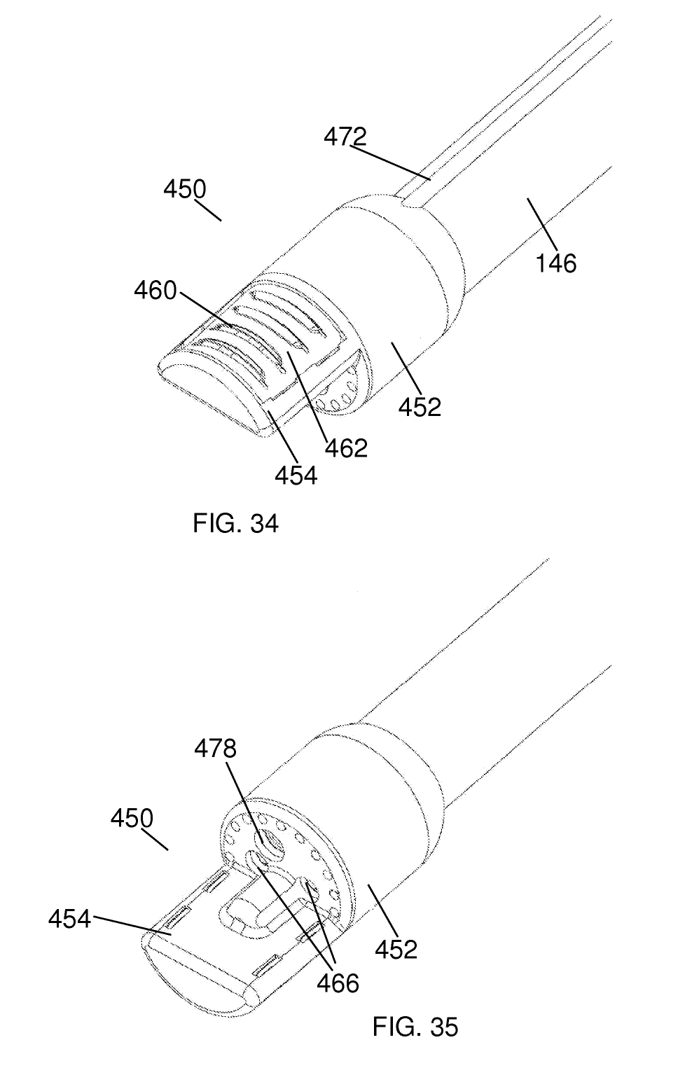

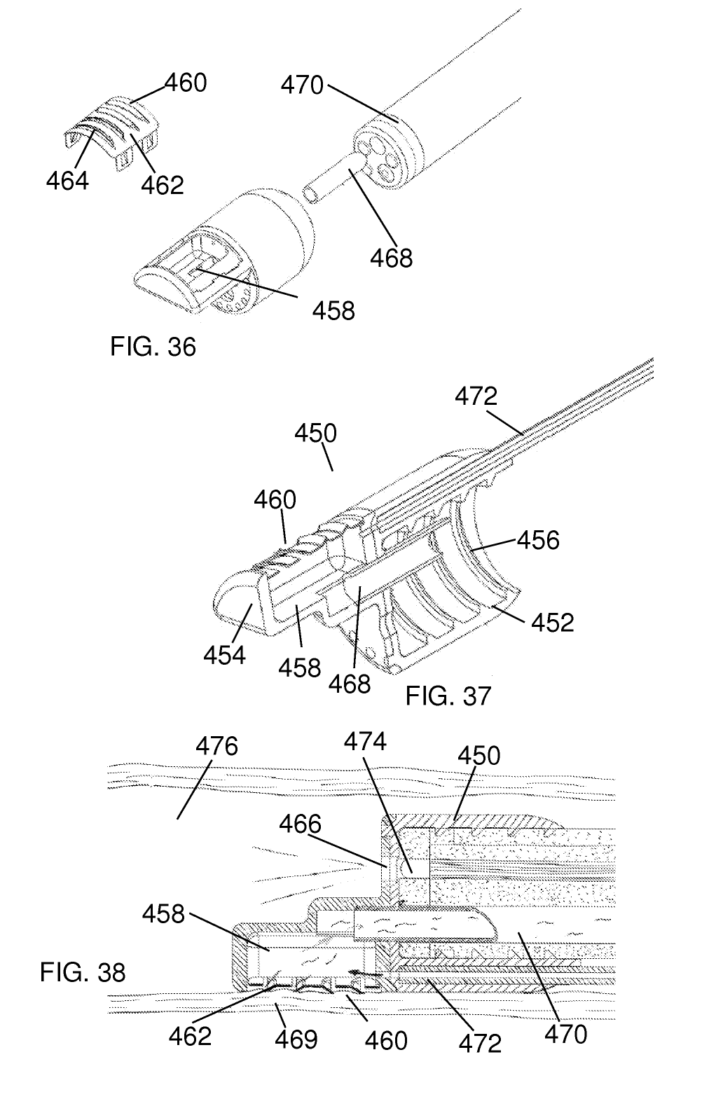

Referring back to the cap or end mounted tissue guard embodiment, the cap 310 is mounted to the distal portion of an access device such as an endoscope. In some instances, it may be advantageous for the cap to cover only a portion of the end surface of the endoscope. For example, the cap may cover the working channel of the scope but not a camera and light surfaces. FIG. 34 shows a cell manipulator 450 that may be a cap mounted on the end of an access device such as an endoscope. The cell manipulator 450 covers the working channel and irrigation channel of the endoscope. The cap may include a relatively soft or elastomeric section 452 and a more rigid portion 454. The cap may be manufactured as two separate components and bonded together or may be manufactured as one piece via methods known in the art such as two-shot molding. Alternatively, the cap may be made of a single material and affixed to the end of the scope via snaps, screws, internal friction o-rings or other methods known in the art. Returning to FIG. 34, the soft portion 452 allows for radial expansion so as to create a friction fit over the distal end of the endoscope. An interior of the soft portion 452 may define circumferential ribs 456 as shown in FIG. 37 so as to create a friction fit with a variety of access devices. As shown in FIG. 37, the rigid portion 454 includes a portion 455 that partially covers a portion 453 of the soft section 452 or in embodiments wherein the soft section does not include a portion 453, the portion 455 may cover the terminal end of the endoscope 146. The rigid portion 454 may be made from any material with suitable rigidity, for example, a thermoplastic or metal. Alternatively, the rigid portion could also be manufactured from an elastomer with properties similar to the soft portion. A clear thermoplastic such as polycarbonate could allow for better visualization of the tissue being manipulated. A portion of the cap forms a chamber 458 as shown in FIG. 36 through which the channels of the endoscope 146 communicate with the fenestrations 460 of the tissue disruption element 462. The cell manipulator 450 has a tissue disruption element 462 that includes of fenestrations 460 with one edge of the fenestration deformed so that the edge protrudes above the level of the fenestration. The protruding edge 464 would be of benefit for cutting/scraping tissue as described later. It is understood that these edges may also be configured so as not to protrude above the fenestrations or could be configured so that the edges protrude inward in a convex manner.

FIG. 35 shows a reverse view of the cell manipulator 450. The cell manipulator 450 has one or more holes or openings 466 which allow a clear view for a camera and a path for light transmission of the endoscope light 474 (FIG. 38). FIG. 36 shows an exploded view of the cell manipulator 450. In this view, a connection tube 468 is shown which may be optionally used to provide a continuous conduit between the suction channel 470 of the endoscope 146 and the chamber 458. The connection tube could be made of metal such as stainless steel or a flexible material such as a thermoplastic elastomer. It should be understood that this connection between the chamber 458 and the endoscope suction channel 470 may be accomplished without the use of the connection tube 468 and alternatively utilizing a gasket or the like for creating a seal. Also shown in FIG. 36, the tissue disruption element 462 may be a separate component that is assembled to the cell manipulator 450 via mechanical methods like snaps, press-fits, bendable tines or by bonding. FIG. 37 is a cross-section view of the cell manipulator 450. In this view, an irrigation tube 472 is shown connecting to the cap via a hole through the soft section 452 of the cap. This irrigation tube communicates to the chamber 458 via a hole through the rigid portion 454 of the cap, to allow active rinsing of the cutting surfaces and for delivery of fluids into the chamber that serves as a carrier of the disrupted cells.

FIG. 38 is a cross-section view showing the use of the cell manipulator 450 mounted to an endoscope 146 in a tissue lumen 476. In this view it may be seen how openings 466 in the cap allow for light transmission into the lumen 476 and visualization by the endoscope camera 478 (FIG. 35). If a clear material were used, a hole may not be necessary for visualization. Fluids may be introduced via the irrigation tube 472 and into the chamber 458. This will aid in prevention of the tissue disruption element 462 from becoming clogged. Suction is applied to the cell manipulator via the suction channel 470 of the endoscope as the scope with the cap is moved axially. As the suction pulls tissue against the tissue disruption element 462 and more specifically the tissue disruption surface cuts/scrapes the tissue 469 and the tissue fragments are evacuated via the chamber 458 and endoscope suction channel 470.

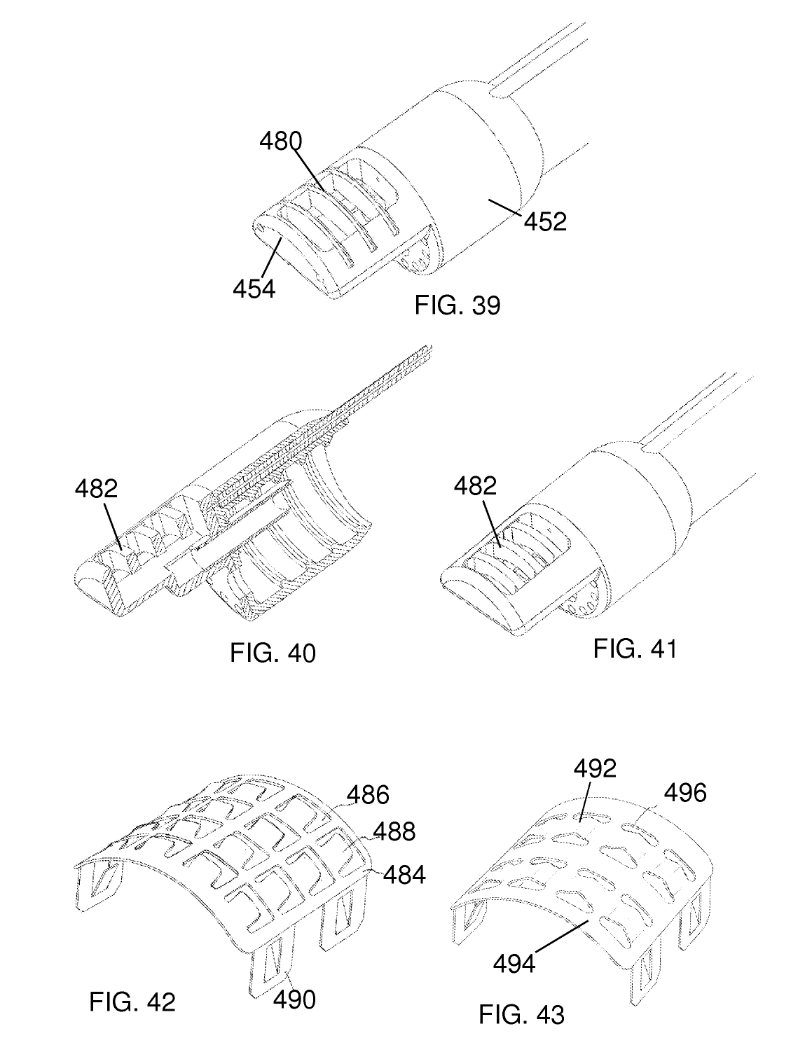

An alternate embodiment of the cell manipulator is shown in FIG. 39. In this embodiment, the tissue disruption elements 480 that are constructed of multiple blades arranged perpendicular to the axis of the cap/scope and spaced axially. The blades may be made of metal, ceramic or other hard materials and attached to the cap via mechanical methods or bonding. The blades have a tissue disruption surface, such as a sharp edge. FIG. 40 is a cross section view of a cap with tissue disruption elements 482 wherein the disruption elements are an integral part of the cap structure. An example of this configuration would be a cap component that is injection molded with the tissue disruption elements 482 molded into the part. FIG. 41 shows a cap of this design mounted to an access devce146.

Referring back to FIG. 36 which illustrates the exploded view of a cap type cell manipulator, a tissue disruption element 462 is shown. An alternate embodiment of a tissue disruption element 484 is shown in FIG. 42. The tissue disruption element 484 includes a plurality of openings or fenestrations 486 arranged in a grid-like fashion. Each fenestration may be associated with one or more tissue disruption surfaces or tangs. In the illustrated embodiment, a tang 488 may be bent upward from the fenestration so as to more effectively cut/scrape the tissue. It is understood that the size, shape, number and arrangement of the fenestrations and tangs may be altered in a number of ways. Connection tabs 490 are shown extending from the tissue disruption element 484 to facilitate assembly to the rigid portion of the cap type cell manipulator. FIG. 43 shows another embodiment of a tissue disruption element 494 including a plurality of fenestrations 496 arranged in a grid-like pattern. The tissue disruption surfaces are metal struts 492, rather than tangs. The metal struts 492 are positioned between adjacent fenestrations and are bent or tented upward, so as to protrude above the level of the fenestrations 496 and better engage the tissue. The cell manipulation device may come in a kit that includes different tissue disruption elements. The surgeon, on-site, may choose which tissue disruption element to employ with the cell manipulator, depending on the application.

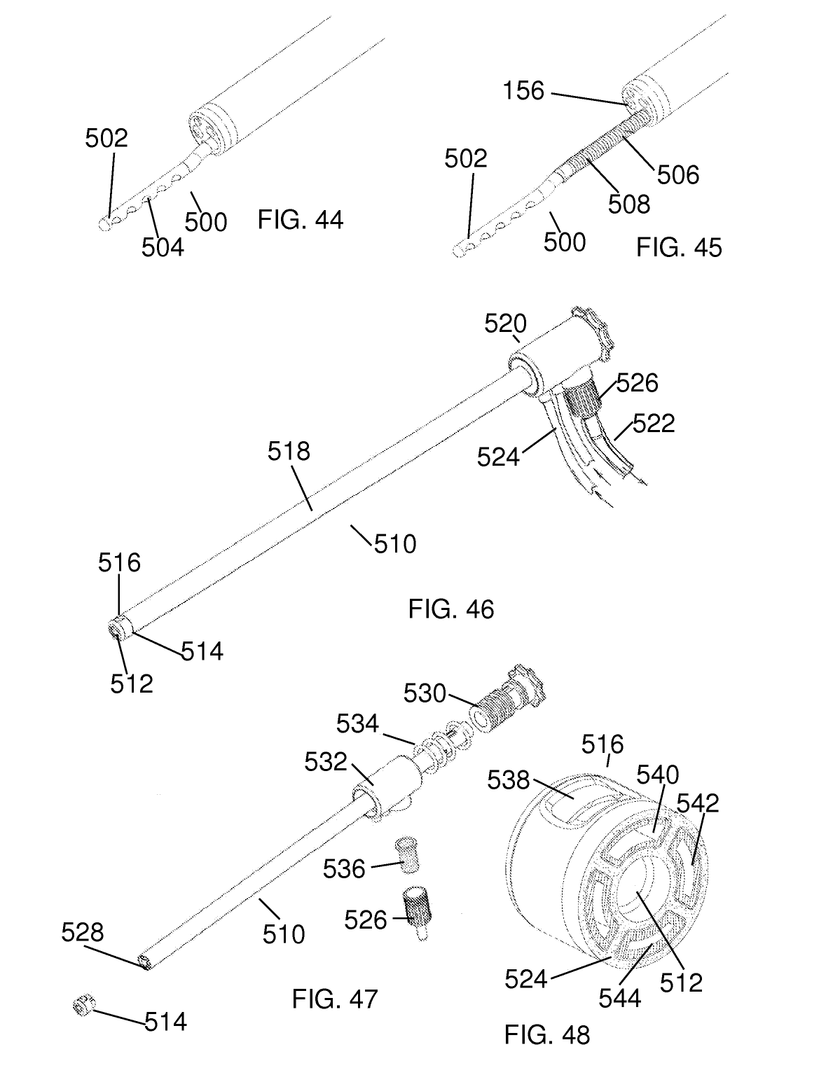

FIG. 44 illustrates a cell manipulator 500 that is inserted through the passageway 156 of access device or endoscope. The distal end portion of the cell manipulator 502 is extended beyond the distal end portion 152 of the access device. The distal portion of the cell manipulator 502 is comprised of a cell disruption element 504 that may be tubular in shape and include a plurality of openings or fenestrations. The cell manipulator 500 is connected to a flexible shaft 506 which may serve to deliver the cell disruption element 504 to a tissue site, and/or also may serve as a conduit for evacuating cell/tissue fragments. In use, suction is applied at the proximal end of the shaft which draws the tissue against the tissue disruption 504 element. Manual or automated movement of the cell manipulator 500 in the axial direction creates a cutting or scraping action to remove layers of tissue/cells. Once the selected cells are disrupted and removed from the esophagus or other body lumen, the cell manipulator is retracted back into passageway 156 of the access device 146. FIG. 45 shows the cell manipulator 500 in the extended position, exposing the flexible shaft. The flexible shaft 506 may be constructed of a flexible tube constructed out of a flexible material such as Nitinol or from metal ribbon wound into a tubular configuration and covered with a sealing layer 508. The sealing layer 508 may be made of flexible plastic tubing and applied in manufacturing via a heat-shrink technique. It is understood that the distal portion 502 of the cell manipulator may be configured to have various bends, shapes, and sizes and that the tissue disruption elements 504 may formed and arranged in various configurations.

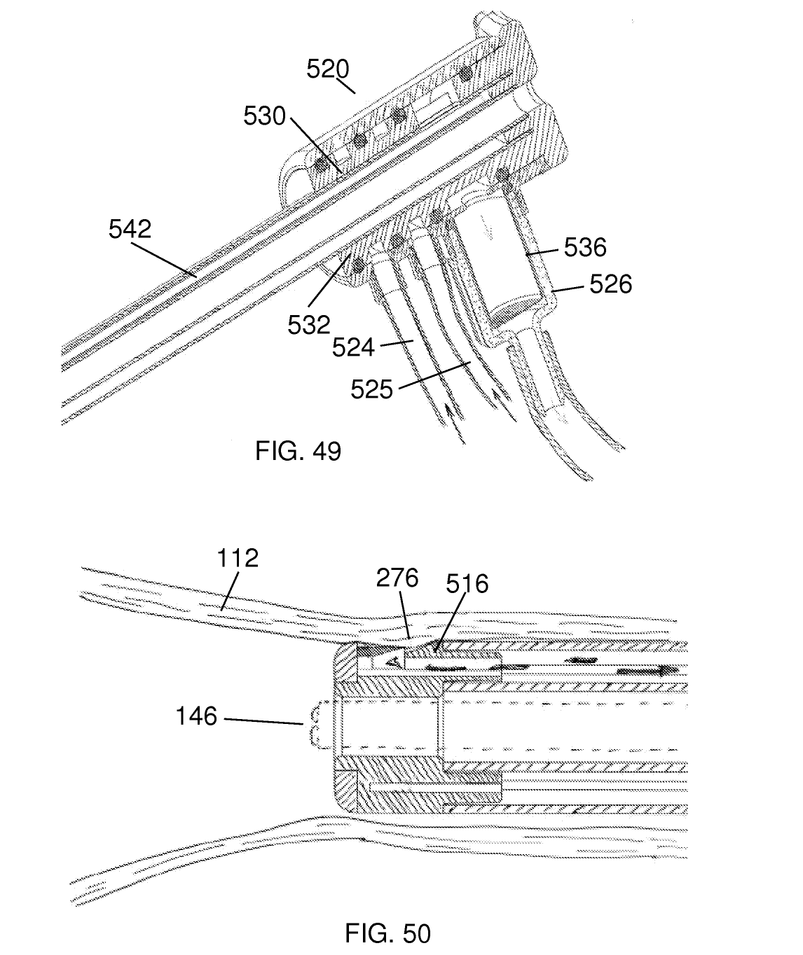

In another embodiment shown in FIG. 46, a cell manipulator 510 is configured to allow passage of an access device through a central channel 512 that that the cell manipulator is delivered or carried over the access device. The overtube cell manipulator 510 has a distal portion 514 with a cell disruption element 516 configured for removal of cells/tissue from the inner wall of the esophagus or other lumen of the body. It is connected to a rigid or flexible shaft that terminates into a manifold. Suction 522 and irrigation 524 tubes are connected to the cell manipulator via the manifold 520. A cell collection basket 526 is also attached to the manifold. FIG. 47 shows an exploded view of the cell manipulator 510 in which the shaft 518 is shown to have multiple lumens 528 which with suction and irrigation tubing on the proximal end. The manifold 520 is shown in two components, the manifold core 530 and the manifold housing 532. Multiple o-rings 534 may provide sealing between the shaft 518, manifold 520 and tubing 525, 522. A cell filter 536 is provided inside the cell collection basket 526 to provide access to harvested tissue and cells. FIG. 48 is a cross section view of the distal portion 514 of the overtube cell manipulator 510. The tissue disruption element 516 is shown over a fenestration 538 leading directly to the primary suction channel 540. Also shown are irrigation channel 542, an optional secondary irrigation channel 542′ as well as an optional auxiliary channel 544. FIG. 49 shows the proximal end of the overtube cell manipulator in cross section. Arrows indicate the direction of fluid and air flow through the device. Two irrigation tubes, the primary irrigation tube 524 and the wash tube 525, are shown connected to the manifold housing 532 through which fluid flows into the manifold core 530 and to the distal portion 514 via the irrigation channels 542.. As tissue/cells are cut/scraped from the esophagus wall, they are evacuated from the distal portion 514, through the channels 542 and into the manifold 520 and out through the collection basket 526 where the tissue/cells are trapped in the cell collection filter 536.

FIG. 50 shows cross-section of the overtube cell manipulator 510 in use inside the esophagus 112 or other body lumen. The cell manipulator 510 is shown inserted over an endoscope 146 (shown in dashed lines). As in the previous cap-style embodiments, tissue 276 is drawn against the tissue disruption element 516 and back-and-forth movement of the manipulator 510 along the axial direction cut/shaves tissue/cells 276 from the lumen wall. Alternatively, tissue disruption elements configured in an axial orientation rather than circumferentially could be utilized in which case the overtube tissue cell manipulator 510 would be rotated in order to create the desired tissue cutting/scraping.

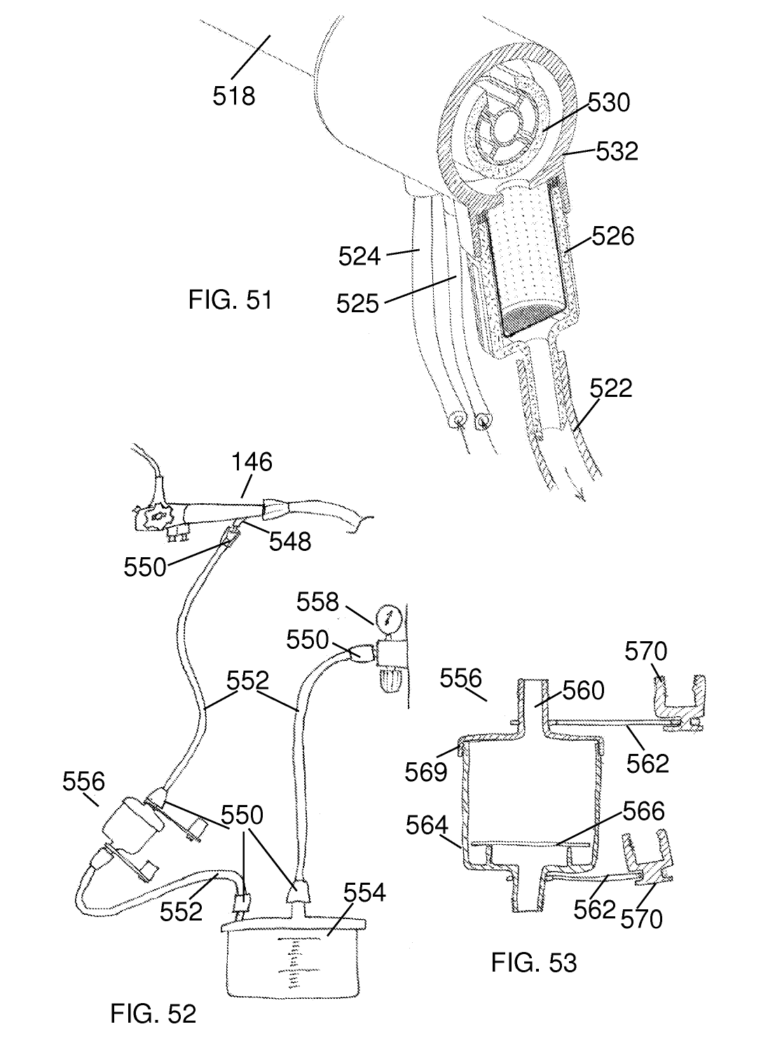

FIG. 51 is a cross section view of the manifold and collection basket shown perpendicular to the axis. This figure illustrates how an internal opening in the manifold core 530 communicates with an opening in the shaft 518 leading into the lumen. Also illustrated is how a hole in the manifold housing 532 may communicate with the collection basket 526.

Turning to the discussion of FIG. 52, it was shown in the embodiment of FIGS. 46-51 that tissue/cells may be removed from the wall of a tissue lumen and suctioned through a device, with the cells/tissue being collected in a collection basket 526. Similarly, with the previous embodiments shown in FIGS. 34-45 or any of the other embodiments disclosed herein, the tissue/cells may be collected. FIG. 52 shows the access device 146 connected to a suction system. The tissue/cells are cut/scraped/suctioned from the tissue lumen wall and travel through the endoscope suction or working channel and out through the suction port 548. A suction hose 552 may be attached to the scope 146 via a hose coupling 550 which carries the tissue/cells and associated fluids back toward a main collection bucket 554. In-line with the tubing 552 is a tissue collection jar 556 which captures and holds the tissue prior to reaching the collection bucket 554.

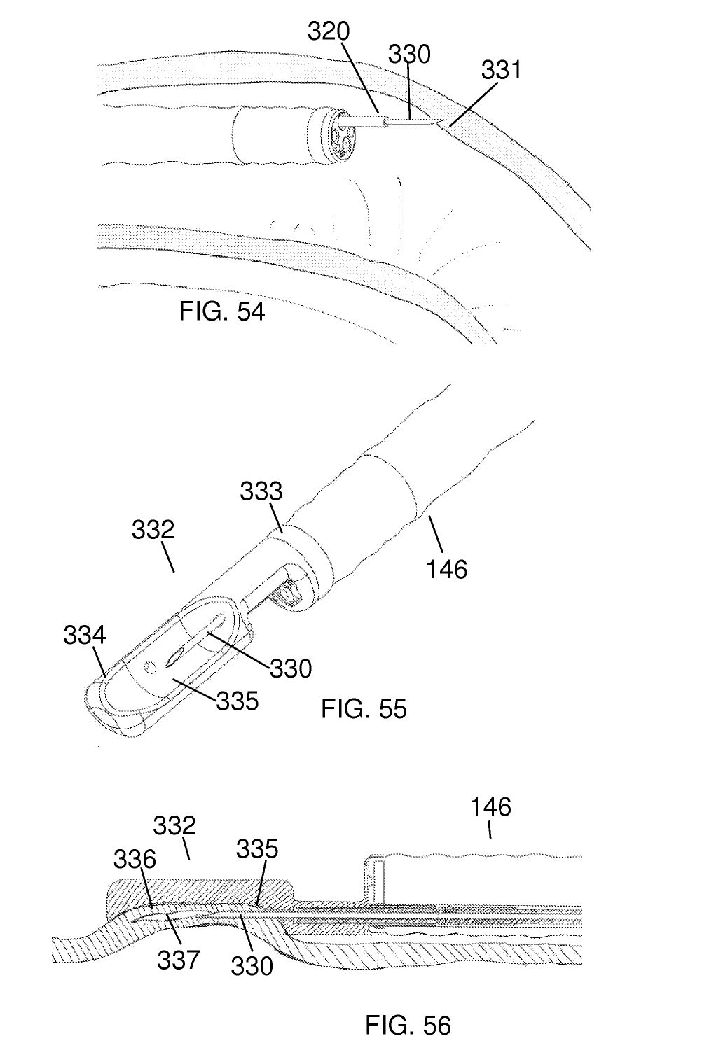

FIG. 53 shows a cross section view of the collection jar 556. The collection jar 556 may utilize a filter or screen 566 to allow fluid to pass while capturing cells/tissue. The collection jar 556 may be composed of a container 564 with a removable lid 560. This embodiment shows the lid 560 attached to the container 564 via threads 569. The removable lid 560 allows access to the inside of the jar so that the tissue/cells may be removed. This collection jar 556 may be easily disconnected from the hose 552 at the couplings 550 and sealed with caps 570 which may be connected via a tether 562 on either end of the jar 556. Prior to sealing, the jar 556 may be filled with a tissue preservative in order to preserve the tissue for pathologic examination at a later time. Alternatively, the tissue/cells and associated fluid may be suctioned directly into the collection bucket 554 without the use of an in-line collection jar 556. The tissue could then be separated from the associated fluids manually. It should be understood that the collection jar 556 does not need to be shaped as shown in FIGS. 52 and 53 to effectively separate the tissue from the fluids. It could be a square, rectangular, oblong or other shapes/configurations and be fashioned as a cassette or cartridge that has a length significantly greater than its width and vice-versa.

It will also be understood that certain modifications may be made by those skilled in the art without departing from the spirit and scope of the subject matter disclosed and/or claimed herein. Thus, the scope of the invention is not limited to the above description, but is set forth in the following claims and/or any future claims made in any application that claims the benefit of this application.

1. An apparatus for removing cells from an inner wall of a gastrointestinal tract, comprising:
a cell manipulator defining an inner chamber and having at least one opening in communication with the inner chamber; and
at least one tissue disruption surface positioned adjacent to the opening,
wherein the tissue disruption surface is configured to separate cells from the inner wall of the gastrointestinal tract.

2. The apparatus of claim 1 wherein the tissue disruption surface includes a sharpened edge.

3. The apparatus of claims 2 wherein the sharpened edge protrudes above the opening.

4. The apparatus of claims 2 wherein the sharpened edge protrudes through the opening and inward toward the chamber.

5. The apparatus of claim 1 wherein the tissue disruption surface and opening are components of a tissue disruption element that is a separate member attached to the cell manipulator.

6. The apparatus of claim 5 wherein the tissue disruption element is removably attached to the cell manipulator.

7. The apparatus of claim 1 further including a suction source associated with the cell manipulator.

8-10. (canceled)

11. The apparatus of claim 1 further including an irrigation mechanism associated with the cell manipulator.

12. (canceled)

13. (canceled)

14. The apparatus of claim 1 further comprising an access device, wherein the cell manipulator is positioned at a distal portion of the access device.

15. (canceled)

16. The apparatus of claim 1 further comprising a cap including the cell manipulator, wherein the cap is positioned at a distal portion of an access device.

17. The apparatus of claim 16 wherein the cap is removably attachable to the distal portion of the access device.

18. The apparatus of claim 1 wherein the cell manipulator is manually or automatically moveable to disrupt tissue.

19. A method of removing cells from an inner wall of a gastrointestinal tract comprising the steps of:
contacting target tissue with a cell manipulator;
applying suction to the target tissue;
disrupting cells from the inner wall of the gastrointestinal tract with the cell manipulator; and
evacuating said cells from the gastrointestinal tract with suction.

20-61. (canceled)

62. An apparatus for removing cells from the inner wall of the gastrointestinal tract, comprising:
a cell manipulator having a closed distal end portion;
a cavity defined by the closed distal end portion;
a side opening in the closed distal end portion, said side opening in communication with the cavity;
a cell disruption surface adjacent the side opening and adapted to access at least a portion of the inner wall through the side opening.

63-82. (canceled)

83. The apparatus of claim 62 wherein the cell disruption element comprises a cell disruption surface.

84. The apparatus of claim 62 wherein the cell disruption element comprises bristles.

85. The apparatus of claim 62 wherein the cell disruption element comprises blades.

86. The apparatus of claim 62 wherein further including a tissue guard.

87. The apparatus of claim 62 further comprising an access device, wherein the cell manipulator is positioned at a distal portion of the access device.

88. The apparatus of claim 62 further including a suction source associated with the cell manipulator.
